(19) United States
(12) Patent Application Publication
(10) Pub. No.: US 2023/0044613 A1
(43) Pub. Date: Feb. 9, 2023

(54) BLOCK COPOLYMERS WITH NITRIC OXIDE DONOR

(71) Applicant: Nanyang Technological University, Singapore (SG)

(72) Inventors: Bee Eng Mary Chan, Singapore (SG); Zheng Hou, Singapore (SG)

(21) Appl. No.: 17/785,863

(22) PCT Filed: Dec. 17, 2020
(86) PCT No.: PCT/SG2020/050755

§ 371 Date: Jun. 15, 2022

(30) Foreign Application Priority Data
Dec. 17, 2019 (SG) 10201912422Q

(51) Int. Cl. A61L 29/08; C09D 153/00; A61L 15/24

(52) U.S. Cl. CPC A61L 29/085; C09D 153/00; A61L 15/24

(57) ABSTRACT

Disclosed herein is a composite material comprising a substrate coated with a block copolymer brush, where the block copolymer brush comprises a first block of a hydrophobic polymer conjugated to a nitric oxide source, where the first block of the hydrophobic polymer is covalently bonded to a surface of the substrate or a first block of a cationic polymer covalently bonded to a surface of the substrate and a second block of a hydrophilic polymer, extending from the first block to form an outer surface of the block copolymer brush.

### FIELD OF INVENTION

The invention relates to a composite material comprising a substrate coated with a block copolymer brush.

### BACKGROUND

The listing or discussion of a prior-published document in this specification should not necessarily be taken as an acknowledgement that the document is part of the state of the art or is common general knowledge.

Medical implants are ubiquitous modern interventions that ameliorate suffering and provide convenience and comfort. However, they are also a major source of infection. Bacteria from patients' microflora can attach to foreign implants and develop into a biofilm form that is recalcitrant to antibiotics. Device-associated infections (DAIs) are a major unsolved healthcare problem that leads to significant morbidity and mortality and large economic costs. Reduction of DAIs through anti-infective surface modification of devices is challenging due to the broad spectrum of bacteria whose colonization and proliferation on device surfaces must be suppressed. It is particularly difficult to suppress Gram-negative bacteria that are intrinsically resistant to many antibiotics. At present, there is no biofilm-resistant coating for major indwelling devices such as catheters and ventilators that can effectively prevent DAI in clinical settings.

Catheters are relatively simple medical devices that are ubiquitous in clinical settings. They are also frequently associated with infections. Some infections, such as central line associated bloodstream infections and catheter-associated urinary tract infection, can be very dangerous. As such, catheters provide a useful test-bed for development and application of anti-infective surface modifications for medical devices. However, the small and long bores of typical catheters mean that their inner surfaces are not easily modifiable in a uniform manner by common techniques (such as plasma activation and UV functionalization).

An ideal biofilm-resistant coating should be antifouling on the device surface against a broad-spectrum of bacteria to reduce bacterial contamination on the surface. It should also have broad-spectrum antibacterial activity in order to kill any bacteria that attaches to the device surface. Moreover, the coated medical device should be non-toxic and non-thrombogenic.

Ideally, the biofilm inhibition effect should persist for as long as the device is implanted. To prevent leaching or solvation of the coating materials or antimicrobial agents into the blood stream or body fluids and for long-term stability, grafting of the coating by covalent linkages to the device surface is preferred as compared to physical adsorption of the coating onto the surface. An effective biofilm-resistant coating which meet the above multi-faceted requirements for clinically useful (30 cm or more) catheter lengths has not been reported.

Antifouling coatings made from purely hydrophilic polymers such as the zwitterionic poly(sulfobetaine methacrylate) (poly(SBMA)) prevent bacteria adhesion but have no antibacterial efficacy. As such, their long-term antibiofilm effect is usually modest (less than 2.0 log₁₀ (99%) inhibition) (Clinical Journal of the American Society of Nephrology 2009, 4 (11), 1787; Angewandte Chemie International Edition 2014, 53 (7), 1746; and Chemistry Communications 2016, 52). While they are not leachable, hydrophilic-cationic coatings may foul through accumulation of dead cells on device surfaces and may also cause blood coagulation.

Silver coatings (such as silver-incorporated nano-hydroxyapatite coating and silver hydrogel-coating) have been tested in vivo but their biofilm inhibition efficacies are generally poor (The Lancet Infectious Diseases 2008, 8 (12), 763; and Journal of Antimicrobial Chemotherapy 2008, 61, 867). Antibiotics are ineffective against many biofilm forming bacteria and current clinical guidelines do not recommend prolonged elution of antibiotics into the body from implanted devices due to the rapid emergence of antimicrobial resistance.

Another category of antibacterial agents is the transiently active Nitric Oxide (NO) gas. NO is an endogenous molecule produced in the body that is involved in various physiological processes such as transmission of neutral messages. NO is both antibacterial and antithrombogenic. NO has a short biological half-life and a small diffusion radius (around 100 μm), as it is rapidly quenched by molecular oxygen or biomolecules (e.g. oxyhaemoglobin). Due to its short half-life, NO exploited for antibiofilm device coating must be generated and released in situ. However, NO-donors are usually toxic. NO-donors employed in anti-infective device coatings are usually blended with the coating material and are leachable which results in toxicity (Journal of Materials Chemistry B 2016, 4, 422; European Journal of Pharmacology 2000, 400 (1), 19; and ACS Biomaterials Science & Engineering 2015, 1, 416). Therefore, NO-donors should ideally be covalently bonded to the surface of the catheter to be non-leachable and non-toxic. Moreover, the concentration of the NO released should be sustained for prolonged periods to provide a long-term (more than 7 days) antibacterial effect, but at a concentration lower than levels known to cause apoptosis.

Known NO donors include diazeniumdiolates (NONOate) and S-nitrosothiols (RSNOs). Small NONOates may release carcinogenic nitrosamine due to their oxidation. Although a coating with covalent linkages to NONOates would not release nitrosamine, the burst release of NO on contact with water is not easily controllable under physiological conditions. This may impair the biocompatibility of the coating and also reduce the lifetime of the antibacterial effect.

Tertiary RSNOs can provide sustained release of NO up to weeks. However, these long-lasting NO donors are usually hydrophobic. Therefore, their presence in a coating will typically increase surface hydrophobicity. This increased hydrophobicity would also promote surface fouling by blood proteins and bacteria. Therefore, there is a need for improved coatings and materials that address one or more problems mentioned above.

### SUMMARY OF INVENTION

It has been surprisingly found that a block copolymer coating as described herein can be applied to a substrate to provide a composite material that has both antimicrobial and antifouling properties. The composite material achieves broad-spectrum and long-term antibiofilm activity. It has negligible toxicity towards mammalian cells and excellent blood compatibility. The formation of the coating is also scalable to coat substrates of clinically useful lengths. The block copolymer coating as disclosed herein may be generally applied to a wide range of medical devices to provide long-term antimicrobial and antibiofilm effects.

Aspects and embodiments of the invention will now be summarised in the following numbered clauses.

1. A composite material comprising a substrate coated with a block copolymer brush, where the block copolymer brush comprises:
a first block of a hydrophobic polymer conjugated to a nitric oxide source, where the first block of the hydrophobic polymer is covalently bonded to a surface of the substrate or a first block of a cationic polymer covalently bonded to a surface of the substrate; and
a second block of a hydrophilic polymer, extending from the first block to form an outer surface of the block copolymer brush.

2. The composite material according to Clause 1, wherein the composite material further comprises a nitric oxide source conjugated to the cationic polymer of the first block.

3. The composite material according to Clause 1 or Clause 2, wherein the nitric oxide source is selected from one or more of a S-nitrosothiol, a NONOate, and a nitric oxide synthase.

4. The composite material according to Clause 3, wherein the nitric oxide source is a S-nitrosothiol and/or a nitric oxide synthase.

5. The composite material according to Clause 4, wherein the nitric oxide source is a tertiary S-nitrosothiol (e.g. a 3-nitroso-tert-thiol-butyl group or a 3-methyl-3-nitrososulfanyl-butyl group).

6. The composite material according to any one of the preceding clauses, wherein the block copolymer brush coating is from 0.1 to 20 μm thick, such as from 5 to 15 μm thick, such as about 10 μm thick.

7. The composite material according to any one of the preceding clauses, wherein the composite material has a contact angle of less than 40° on a surface of the substrate coated with the block copolymer brush, optionally wherein the composite material has a contact angle of less than 20°, such as from 5 to 19°, on a surface of the substrate coated with the block copolymer brush.

8. The composite material according to any one of the preceding clauses, wherein the substrate is formed from a polymeric material, optionally wherein the substrate is selected from one or more of the group consisting of polyurethane, silicone, latex, and polyester polyurethane.

9. The composite material according to Clause 8, wherein the substrate is formed from polyurethane.

10. The composite material according to any one of the preceding clauses, wherein the first block is formed from a polymer comprising one or more vinylogous monomers, where at least one of the one or more vinylogous monomers contains a functional group suitable to conjugate to a nitric oxide source.

11. The composite material according to Clause 10, wherein the functional group suitable to conjugate to a nitric oxide source is selected from one or more of the group consisting of an amine, a hydroxyl, a carboxylic acid, a carboxylic acid ester, and an epoxide.

12. The composite material according to Clause 10 or Clause 11, wherein the one or more vinylogous monomers are selected from the group consisting of an acrylate, an alkylacrylate (e.g. a methacrylate), an acrylamide, an alkylacrylamide (e.g. a methacrylamide), an acrylic acid, an alkyl acrylic acid, and an allylic monomer compound.

13. The composite material according to Clause 12, wherein the one or more vinylogous monomers are selected from the group consisting of allyl amine, amino ethyl methacrylate (AEMA), hydroxyl ethyl methacrylate (HEMA), hydroxylethyl acrylate (HEA), hydroxyl ethyl acrylamide (HEAA), hydroxypropyl methacrylate (e.g. 2-hydroxypropyl methacrylate (HPMA)), hydroxybutyl acrylate, N-[Tris(hydroxymethyl)methyl] acrylamide, allyl alcohol, acrylic acid, methacrylic acid, glycidyl methacrylate (GMA), and ally glycidyl ether (AGE).

14. The composite material according to any one of Clauses 10 to 13, wherein the first block is formed from a polyacrylate, optionally wherein the first block is formed from one or more monomers selected from the group consisting of hydroxyethyl methacrylate, hydroxyethyl acrylate, hydroxypropyl-methacrylate, and hydroxybutyl acrylate.

15. The composite material according to any one of the preceding clauses, wherein the second block is formed from a polymer comprising one or more vinylogous hydrophilic monomers.

16. The composite material according to Clause 15, wherein the one or more vinylogous hydrophilic monomers are selected from the group consisting of a hydrophilic acrylate, a hydrophilic alkylacrylate (e.g. a hydrophilic methacrylate), a hydrophilic acrylamide, a hydrophilic alkylacrylamide (e.g. a hydrophilic methacrylamide), a hydrophilic acrylic acid, a hydrophilic alkyl acrylic acid, and a hydrophilic allylic monomer compound.

17. The composite material according to Clause 16, wherein the one or more vinylogous hydrophilic monomers are selected from the group consisting of methyl terminated poly(ethylene glycol) methacrylate, methyl terminated poly(ethylene glycol) acrylate, allyl poly(ethylene glycol), oligoethyleneglycol methacrylate, a zwitterionic monomer (e.g. [2-(methacryloyloxy) ethyl] dimethyl-(3-sulfopropyl) ammonium hydroxide (SBMA), methacryloyloxyethyl phosphorylcholine (e.g. 2-methacryloyloxyethyl phosphorylcholine (MPC)), N-(3-methacryloylimino) propyl-N,N-dimethylammonio propanesulfonate (SBMAm), 3-(2′-vinyl-pyridinio) propanesulfonate (VPPS), carboxybetaine methacrylate (CBMA), sulfobetaine methacrylate, sulfobetaineacrylamide, 3-[(3-acrylamidopropyl) dimethylammonio] propanoate, and 3-[[2-(methacryloyloxy) ethyl] dimethylammonio] propionate), and a vinylogous-modified polysaccharide (e.g. dextran methacrylate, hyaluronate methacrylate).

18. The composite material according to Clause 16 or Clause 17, wherein the second block is formed from one or more monomers selected from the group consisting of oligoethyleneglycol methacrylate, methacryloyloxyethyl phosphorylcholine, sulfobetaine methacrylate, sulfobetaineacrylamide, 3-[(3-acrylamidopropyl) dimethylammonio] propanoate, and 3-[[2-(methacryloyloxy) ethyl] dimethylammonio] propionate.

19. The composite material according to any one of Clause 1 and Clauses 3 to 18, wherein:
the first block is formed from poly(hydroxyethyl methacrylate);
the second block is formed from poly(sulfobetaine methacrylate); and
the nitric oxide source is a tertiary S-nitrosothiol.

20. A method of forming a composite material as described in any one of Clauses 1 to 19, the method comprising the steps of:

(a) providing a substrate coated with a polymer brush, where the polymer brush is formed from a hydrophobic or cationic polymer covalently bonded to a surface of the substrate;

(b) reacting the hydrophobic or cationic polymer in the polymer brush with a hydrophilic monomer in a polymerisation reaction to form a block copolymer brush; and

(c) conjugating a nitric oxide source to the hydrophobic or cationic polymer of the block copolymer brush.

21. A method of forming a composite material as described in any one of Clauses 1 to 19, said method comprising the steps of:

(ai) providing a substrate coated with a block copolymer brush, where the block copolymer brush is formed from:
a first block of a hydrophobic or cationic polymer covalently bonded to a surface of the substrate; and
a second block of a hydrophilic polymer, extending from the first block to form an outer surface of the block copolymer brush; and

(bi) conjugating a nitric oxide source to the hydrophobic or cationic polymer of the first block.

22. An article formed from a composite material as described in any one of Clauses 1 to 19, wherein a substrate of the composite material has at least one surface that is coated with a block copolymer brush as described in any one of Clauses 1 to 19.

23. The article according to Clause 22, wherein the article is a catheter formed from the composite material, where the catheter has an outer surface and an inner surface, and one or both of the outer surface and inner surface have been coated with a block copolymer brush as described in any one of Clauses 1 to 19.

24. The article according to Clause 22, wherein the article is a wound dressing formed from the composite material, where the wound dressing has an outer surface and an inner surface, and the inner surface is coated with a block copolymer brush as described in any one of Clauses 1 to 19.

### BRIEF DESCRIPTION OF DRAWINGS

FIG. 1 Illustrations and synthesis scheme of antifouling and nitric oxide (NO) emitting diblock copolymer brush ((#10) H(N)-b-S) grafted from PU catheter: (A) The diblock copolymer with a subsurface NO-emitting block modified from poly(HEMA) (H) with a RSNO (N) and a surface block of antifouling poly(SBMA) (S). (B) Flow reactor for surface modification of long catheter with long narrow lumen (grey arrows: flow of monomer solution). (C) Synthesis scheme of diblock copolymer (#10) H(N)-b-S coating via ozone pre-treatment of PU followed by surface-initiated RAFT diblock copolymerization. (CTA is chain transfer agent of 4-Cyano-4-(phenylcarbonothioylthio) pentanoic acid (CPCPA), ACVA is the thermal initiator 4,4′-Azobis(4-cyanopentanoic acid)).

FIG. 2 Characterization of (#10) H(N)-b-S coating: (A) Visual appearance of long (i) (#1) unmodified and (ii) (#10) H(N)-b-S coated catheters. (B) FTIR-ATR characterization of steps in the synthesis of (#10) block copolymer coating: (i) unmodified PU control, (ii) first block of poly(HEMA), (iii) after second block copolymerization to make (#7) H-b-S, (iv) reaction of RSNO-Cl with subsurface poly(HEMA) block to get (#10) H(N)-b-S. (C) SEM characterizations (scale bar=10 μm): surface and cross section (inset) of (i) (#1) unmodified PU and (ii) (#10) H(N)-b-S. (D) 3D AFM characterization of (i) unmodified PU catheter and (ii) (#10) H(N)-b-S. (E) Water contact angle change of (#10) H(N)-b-S over 1-month incubation in water. (F) HPLC detection of NO release precursor (NTMB-Cl) leaching to different solvents (N.D. refers to no detection of leaching). (G) NO release flux of (#10) H(N)-b-S coating at 37° C. over 15 days.

FIG. 3 (A) 24 hr-antibiofilm efficacy of coatings measured by static media model against broad-spectrum Gram-negative and Gram-positive bacteria. The general log reduction of #10 is compared with other catheter coating controls by Student's t-test, ****P<0.0001, ***P<0.001, **P<0.01, *P<0.05. (B) Long-term (30 Days) intraluminal antibiofilm effect against (i) MRSA and (ii) P. aeruginosa bacteria.

FIG. 4 Biocompatibility and hemocompatibility of coatings: (A) Antithrombogenic effect of coatings measured by platelet activation and thrombus formation. (B) SEM images of thrombus formed on catheters (Left: before incubation with rabbit whole blood. Right: after 2 hrs incubation with rabbit whole blood) (i) (#1) unmodified catheter, (ii) (#10) H(N)-b-S (scale bar=10 μm). (C) Activation of blood immune cells. Student's t-test of #10 against blood w/o treatment, n.s P>0.05. (D) Blood protein fouling on catheters after 24 hrs incubation with protein or serum. Student's t-test of #10 against unmodified catheter, ****P<0.0001, **P<0.01.

FIG. 5 In vivo antibacterial efficacy of coated catheters: (A) Murine subcutaneous 24 hrs implantation infection model (i) illustration of subcutaneous implantation and infection, (ii) antibiofilm efficacy of catheters (#8, #9 and #10) against antibiotic-sensitive and multi-drug resistant (MDR) bacteria, (iii) appearance of MDR PAER infected subcutaneous pockets after 24 hrs implantation; (a) pus (black circle) found in the subcutaneous pocket implanted with (#1) unmodified catheter (b) minimal pus found in the subcutaneous pocket implanted with (#10) H(N)-b-S catheter. The general log reduction of #10 is compared with other catheter coating controls by Student's t-test, (#2) silver catheter, ****P<0.0001, (#8) catheter, ***P<0.001, (#9) catheter, **P<0.01. (B) Porcine central venous catheter model with MRSA: (i) illustration of catheter implantation in porcine jugular vein and infection of Pig III, (ii) Biofilm (arrow, and sub-figure) and thrombus formation (left arrow) on catheters observed under SEM (scale bar=50 μm) with (a) (#1) unmodified PU catheter and (b) (#10) H(N)-b-S catheter. (iii) antibiofilm efficacy of catheter (#10) H(N)-b-S.

FIG. 6 Synthesis of (A) homo poly(SBMA) coating ((#3) S) and (B) homo poly(HEMA-NO) coating ((#4) H(N)).

FIG. 7 Synthesis of random copolymer coating ((#6) H-r-S and (#9) H(N)-r-S).

FIG. 8 Synthesis of crosslinked coating ((#5) H-x-S and (#8) H(N)-x-S).

FIG. 9 Further characterization of (#10 H(N)-b-S) (A) characterization of poly(HEMA) formed in the first step in the synthesis of (#10) H(N)-b-S: (i) SEM image of catheter surface and cross section (inset) (scale bar=10 μm), (ii) AFM image of surface morphology with measured root mean square height variation. (B) contact angle of poly(HEMA). (C) Quantification of NO-release precursors on (#10) H(N)-b-S: (i) NO flux profile at 55° C., (ii) Cumulative NO released.

FIG. 10 Stability of coatings. Long-term hydrophilicity under various conditions: (A) PBS saline (B) Serum (C) S. aureus inoculum (D) P. aeruginosa inoculum. The contact angles are shown in the insets ((i) (#1) unmodified PU, (ii) (#3) S, (iii) (#10) H(N)-b-S), the dotted line in the graphs indicates 10°.

FIG. 11 (A) SEM and AFM measurements of surface morphology and cross-sections of catheters. (B) Contact angle measurements of catheters

FIG. 12 Characterization of coating (#8) H(N)-x-S. (A) NO flux measured at 55° C., (B) Cumulative NO released.

FIG. 13 Characterization of coating (#9) H(N)-r-S. (A) NO flux measured at 55° C., (B) Cumulative NO released.

FIG. 14 Acute (2 hrs) antimicrobial efficacy measured by the reduction of bacteria loaded on surface.

FIG. 15 (A) In vitro antibiofilm efficacy of intermediate coatings (#5, #6 and #7) against some Gram-positive and Gram-negative bacteria. Student's t-test, n.s P>0.5, (B) In vitro antibiofilm efficacy of NO-releasing coatings against multi-drug resistance (MDR) Gram-negative bacteria. Student's t-test, ***P<0.001, **P<0.01. (C) Fluorescence Microscopy images of catheters incubated with MRSA and P. aeruginosa (scale bar=20 μm).

FIG. 16 Illustration of intraluminal circulation setup for antibiofilm test.

FIG. 17 In vitro mammalian cell compatibility of extractants from modified catheters soaked in DMEM for (A) 24 hrs and (B) 72 hrs following 15010993-5. (C) Hemocompatibility of intermediate catheters (#5, #6 and #7) measured by platelet activation and thrombus formation. (D) Activation of blood immune cells. (E) Blood protein fouling on catheters after 24 hrs incubation with protein or serum.

FIG. 18 Monitoring of pigs' physiological parameters (time point at 0 denotes point of completion of surgery to implant the catheter(s).). (A) Long-term observation (i.e. when the effects of anaesthesia are not present), (i) mean arterial pressure (MAP), (ii) heart rate (HR) (N.D. means not done because infected pig was sacrificed on Day 5). (B) Observation after surgery until pig recovered from anaesthesia, (i) mean arterial pressure (MAP), (ii) heart rate (HR).

FIG. 19 Characterizations of surface coatings. (A) Contact angles; (B) roughness; and (C) long term NO release flux.

FIG. 20 (A) Antifungal efficacy against Candida albicans. (B) Biofilm fungi inhibition efficacy against Candida albicans.

FIG. 21 SEM images of thrombus formed on catheters, Left: before incubation with rabbit whole blood. Right: after 2 hrs incubation with rabbit whole blood: (I) commercial silver coated catheter.

FIG. 22 Long term hydrophilic test in water: (i) unmodified PU (ii) #3 S (iii) #10 H(N)-b-S.

FIG. 23 HPLC detection of NO release precursor leached or extracted into different solvents after different time periods. N.D means no detection of leaching (intensity=0). (A) 24 hrs extraction (B) 1-week extraction.

FIG. 24 (SEM images of thrombus formation on catheter samples (left: before incubation with whole rabbit blood; right: after 2 hrs incubation with whole rabbit blood; Circle indicates partial coverage of thrombus).

FIG. 25 In vivo antibacterial efficacy (A) Murine subcutaneous model (i) In vivo antibacterial efficacy. (ii) Images of infected subcutaneous pocket and recovered catheter after 24 hrs implantation. (a) Serious sign of infection found in the subcutaneous pocket implanted with unmodified catheter (b) Minimal sign of infection found in the subcutaneous pocket implanted with #10 H(N)-b-S catheter. (c) Clotted blood found on unmodified catheter. (d) No blood clotting found on #10 H(N)-b-S catheter. (B) Porcine haemodialysis model (i) Biofilm and thrombus formation on catheters observed using SEM (Scale bar=50 μm). (a) unmodified catheter (b) #10 H(N)-b-S catheter. (ii) Quantified biofilm bacteria on surface of unmodified catheter and #10 H(N)-b-S.

FIG. 26 Body temperature (A) and heartbeat rate (B) monitored for porcine haemodialysis catheterization model

FIG. 27 Synthesis of an electrostatic LBL coating (#30) and a covalent LBL coating (#40).

FIG. 28 FTIR-ATR characterization of (#30) coating.

FIG. 29 FTIR-ATR characterization of (#40) coating.

FIG. 30 Scanning Electronic Microscopy images showing (i) cross-sections and (ii) surface morphology of catheters: A) PU control; B) an ozone-treated PU with cationic coating; C) (#40) coating; and D) (#30) coating.

FIG. 31 Illustration of the antifouling and excellent blood compatibility of the block copolymer brush ((#10) H(N)-b-S) grafted from PU catheter.

### DESCRIPTION

It has been surprisingly found that a block copolymer coating can be applied to a substrate surface to provide a composite material that has both anti-fouling and antimicrobial properties. This composite material requires a first polymeric block attached to the substrate surface formed from a cationic polymer, a polymer functionalised with nitric oxide (NO) releasing components, or a combination of both. The composite material also requires a second polymeric block that extends from the first polymeric block to form a surface of the composite material. Without wishing to be bound by theory, it is believed that the first polymeric block provides the composite material with its desirable antimicrobial/bactericidal properties, while the second polymeric block provides the composite material with a highly hydrophilic and antifouling surface. It is noted that the covalent immobilization of a NO donor with precise control of the donor's depth within a hydrophilic coating has not been reported.

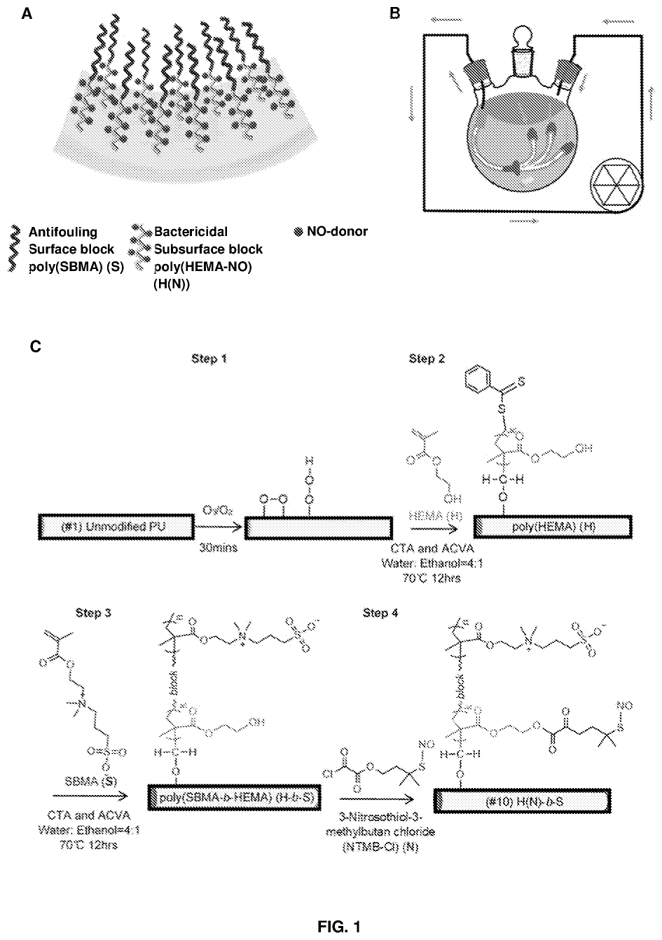

FIG. 1A discloses an example of the composite materials disclosed herein. FIG. 1A relates to a uniform, high-density precision diblock copolymer brush (termed H(N)-b-S) consisting of a surface block of antifouling poly(sulfobetaine methacrylate) (labeled as S) with a subsurface block of bactericidal RSNO-modified poly(hydroxyethyl methacrylate) (labeled as H(N)) (FIG. 1A). This material is covalently “grafted from” a slender polyurethane (PU) catheter and shows outstanding broad spectrum antibiofilm efficacy in vitro and in vivo. The brush also has good antithrombogenicity and biocompatibility. In addition, it is noted that the formation of the composite materials can be readily scaled-up. As an example of this, (FIG. 1B) discloses a flow reactor that has been used to coat a 30 cm long catheter with the H(N)-b-S coating.

Thus, in a first aspect of the invention, there is provided a composite material comprising a substrate coated with a block copolymer brush, where the block copolymer brush comprises:
a first block of a hydrophobic polymer conjugated to a nitric oxide source, where the first block of the hydrophobic polymer is covalently bonded to a surface of the substrate or a first block of a cationic polymer covalently bonded to a surface of the substrate; and
a second block of a hydrophilic polymer, extending from the first block to form an outer surface of the block copolymer brush.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

When used herein, the term “substrate” refers to any material with a surface to which a block copolymer brush can be affixed to through covalent bonding. Examples of suitable materials include any polymeric material that may conjugate directly with the monomeric materials used in the first block or it may be a polymeric material whose surface has been activated by a suitable surface activation method. Examples of suitable surface activation methods include, but are not limited to ozonolysis and plasma treatments. In embodiments of the invention that may be mentioned herein, the substrate may be formed from a polymeric material. In more particular embodiments of the invention that may be mentioned herein, the substrate may be selected from one or more of the group consisting of polyurethane, silicone, latex, and polyester polyurethane. For example, the substrate may be a polyurethane.

The substrate material may also be presented in any suitable form. For example, the substrate material may be presented as a film, a sheet material, a tubular material, a foam surface, and the like. As will be appreciated, it is possible for the coating to be applied to a single surface of a substrate, but it is also possible for two or more surfaces of a substrate to be coated. For example, if the substrate is tubular in nature, the inner and outer surfaces of the tube may be coated.

When used herein, the term “polymer brush”, or more specifically in this case, “block copolymer brush” refers to a coating consisting of polymers tethered to a surface of a substrate material. As will be appreciated, a copolymer is formed by the polymerisation of two or more different monomers together. This may be done in a random fashion (e.g. the polymer chain is formed from a random dispersion of the monomers) or, as is the case here, it may be formed as a block copolymer that comprises two or more subunits. Each subunit in a block copolymer may be formed from a homopolymeric material or a copolymeric material.

When used herein, the term “homopolymeric material” used as a block of the current invention may refer to a polymeric material that is formed only from a single monomeric material. This may be achieved by conducting the polymerisation reaction in isolation in the absence of any other monomeric materials.

When used herein, the term “copolymeric material” used as a block of the current invention may refer to a polymeric material that is formed from two or monomeric materials that have similar properties (e.g. they are both highly hydrophilic, or they may be both cationic). This may be achieved by conducting the polymerisation reaction in isolation in the absence of any other monomeric materials.

As it may be desired to form the composite material in a one-pot process, it is possible for the first and second blocks to include trace amounts of the monomers used to form the opposite block. In addition, in such one-pot processes, there may be a third block in between the first and second blocks which is a copolymer formed from the monomers used in both the first and second blocks.

Thus, it is possible:
for the homopolymer to also include a small fraction of repeating units derived from the other block's monomeric materials. For example, the first block may include a small fraction of the second block's monomer(s) and/or the second block may include a small fraction of the first block's monomer(s) (as will be appreciated, at least one of the first block and the second block will be intended to use only a single monomer, so as to provide a substantially homopolymeric block). When a small fraction of the other block's monomeric material is present, this will be less than or equal to 10 mol %, such as less than or equal to 5 mol %, such as less than or equal to 1 mol %, such as less than or equal to 0.1 mol %, such as less than or equal to 0.01 mol %; and
for the copolymer to also include a small fraction of repeating units derived from the other block's monomeric material(s). For example, the first block may include a small fraction of the second block's monomer(s) and/or the second block may include a small fraction of the first block's monomer(s). When a small fraction of the other blocks monomeric material is present, this will be less than or equal to 10 mol %, such as less than or equal to 5 mol %, such as less than or equal to 1 mol %, such as less than or equal to 0.1 mol %, such as less than or equal to 0.01 mol %.

In the current invention, the block copolymer brush is formed from a first block of a hydrophobic polymer conjugated to a nitric oxide source or a cationic polymer (the latter unconjugated or conjugated to a nitric oxide source) covalently bonded to a surface of the substrate and a second block of a hydrophilic polymer, extending from the first block (directly or indirectly via the presence of a third block, which is a copolymeric block formed from the monomers used to make the first and second blocks) to form an outer surface of the block copolymer brush. As described herein, it is believed that this arrangement has a number of advantages. In particular, this arrangement provides an outer surface that has a low contact angle (with water). In other words, the outer surface of the block copolymer brush is hydrophilic. Moreover, the block copolymer brush is chemically stable and its outer surface remains hydrophilic over a prolonged period of time (such as over a month). The hydrophilicity of the outer surface is not affected even when a nitric oxide source, which is typically hydrophobic, is conjugated to the first block of the block copolymer brush. Without wishing to be bound by theory, it is believed that the hydrophilicity of the block copolymer brush on its outer surface enables said brush to achieve superior broad-spectrum antibiofilm activity with good biocompatibility and low fouling.

The antimicrobial/antibacterial efficacy of the composite materials disclosed herein may be achieved using a nitric oxide source or a cationic polymer, or a combination of both. The composite material disclosed herein displays antimicrobial/antibacterial properties. Without wishing to be bound by theory, it is believed that these antimicrobial/antibacterial properties are derived from the first block of the block copolymer brush. There are three possible arrangements of the first block contemplated herein:
a first block formed from a hydrophobic polymer conjugated to a nitric oxide source;
a first block formed from a cationic polymer; and
a first block formed from a cationic polymer that has then been conjugated to a nitric oxide source.

Without wishing to be bound by theory, it is believed that the addition of a nitric oxide source to the composite material enables said material to achieve short-term and long-term antibiofilm efficacy against a broad spectrum of bacteria with excellent blood compatibility. It has been surprisingly found that incorporating a nitric oxide source in a block copolymer coating as described herein enables the antibacterial property of NO emission to be exploited while avoiding the fouling side effects of the nitric oxide source at the coating surface.

The term “conjugated” when used herein refers to a covalent bond formed between the nitric oxide source and the hydrophobic polymer or cationic polymer of the first block. Thus, as will be appreciated, the first block will be formed from (one or more) monomeric material(s) which may contain a functional group that may be used to form a covalent bond (see below for non-limiting examples) with the nitric oxide source. For example, the hydrophobic polymer may contain a functional group selected from one or more of the group consisting of an amine, a hydroxyl, a carboxylic acid, a carboxylic acid ester, and an epoxide. The cationic polymer may contain the same functional groups, or it may contain a cationic group that can itself act as the group that conjugates to the nitric oxide source. For example, the cationic group may contain a cationic amino group that may be able to conjugate to the nitric oxide source. Examples of said polymers will be discussed in more detail below.

When used herein, the term “nitric oxide source” is intended to relate to a moiety containing a functional group that can undergo reaction to release nitric oxide (NO) when exposed to suitable conditions, as well as a moiety that contains a material that can generate NO through its action on a substrate material found in the environment that the composite material is exposed to. Examples of a functional group that can undergo reaction to release NO include, but are not limited to compounds that contain an S-nitrosothiol or a NONOate group. Examples of a material that can generate NO through its action on a substrate material found in the environment that the composite material is exposed to include, but is not limited to NO synthases. Thus, in embodiments of the invention, the nitric oxide source may be a S-nitrosothiol, a NONOate, a NO synthase (e.g. iNOS) and combinations thereof. In more particular embodiments of the invention that may be mentioned herein, the nitric oxide source may be a S-nitrosothiol and/or a nitric oxide synthase.

Examples of S-nitrosothiols that may be mentioned herein include, but are not limited to, tertiary S-nitrosothiols. Tertiary S-nitrosothiols that may be mentioned herein include, but are not limited to a 3-nitroso-tert-thiol-butyl group or a 3-methyl-3-nitrososulfanyl-butyl group. Examples of a nitric oxide synthase include, but are not limited to an inducible NOS (iNOS).

As will be appreciated, the covalent bond formed between the polymer of the first block and the nitric oxide source may be direct (e.g. the covalent bond may be formed between the functional group that may be used to form a covalent bond of the hydrophobic or cationic polymer of the first block with the nitric oxide source and/or enzyme without use of any linking moiety) or it may be indirect (e.g. a linking moiety attached to the functional group that may be used to form a covalent bond of the hydrophobic or cationic polymer of the first block with the nitric oxide source and/or enzyme). Further details concerning the indirect arrangements are included in the examples below.

The nitric oxide source may be covalently bonded to the first block of the block copolymer brush without reaction with the second block in a post-modification process via chemistry that includes, but is not limited to:
1) esterification of surface hydroxyl groups by reaction with acyl chloride;¹
2) Schiff base reaction between surface amino groups with free aldehydes;²
3) ring-opening reaction between surface epoxy groups with primary amines;³
4) urethane bond formation between surface hydroxyl groups with free isocyanide groups;⁴ and
5) amide bond formation of surface acid groups with primary amines.⁵ (1) Guangqun Zhai, W. H. Y., E. T. Kang, K. G. Neoh. Functionalization of Hydrogen-Terminated Silicon with Polybetaine Brushes via Surface-Initiated Reversible Addition-Fragmentation Chain-Transfer (RAFT) Polymerization. Industrial & Engineering Chemistry Research 2004, 43, 1673.(2) M. Alagem-Shafir; E. Kivovich; Tzchori; N. Lanir; M. Falah; M. Y. Flugelman; U. Dinnar; R. Beyar; N. Lotan; Sivan, S. S. The formation of an anti-restenotic/anti-thrombotic surface by immobilization of nitric oxide synthase on a metallic carrier. Acta Biomaterialia 2014, 10, 2304.(3) Suresh Kumar Raman Pillai, S. R., Yogesh Vikhe, Hou Zheng, Chong Hui Koh, Mary B. Chan-Park. Novel Antimicrobial Coating on Silicone Contact Lens Using Glycidyl Methacrylate and Polyethyleneimine Based Polymers. Macromolecular Rapid Communication 2020.(4) Merve Gultekinoglu; Yeliz Tunc Sarisozen; Ceren Erdogdu; Meral Sagiroglu; Eda Ayse Aksoy; Yoo Jin Oh; Peter Hinterdorfer; Kezban Ulubayram. Designing of dynamic polyethyleneimine (PEI) brushes on polyurethane (PU) ureteral stents to prevent infections. Acta Biomaterialia 2015, 21, 44.(5) Hana Vaisocherová; Wei Yang; Zheng Zhang; Zhiqiang Cao; Gang Cheng; Marek Piliarik; Jiří Homola; Jiang, S. Ultralow Fouling and Functionalizable Surface Chemistry Based on a Zwitterionic Polymer Enabling Sensitive and Specific Protein Detection in Undiluted Blood Plasma. Analytical Chemistry 2008, 80 (20), 7894.

The above chemistries thus form covalent bonds to a functional group present in the hydrophobic polymer or on the cationic polymer used to form the first block.

Given that the composite material is formed by grafting (i.e. covalently attaching) the block copolymer brush coating onto the surface of a substrate material, the resulting coating may be thicker and denser that may otherwise be achievable by other methods (see below for details of the synthesis of the composite material). For example, the block copolymer brush coating may have a thickness of from 0.1 to 20 μm, such as from 5 to 15 μm, such as about 10 μm. Furthermore, the first block of a hydrophobic or cationic polymer may provide a thickness of from 1 to 8 μm, such as from 3 to 5 μm. As will be appreciated, the second block of a hydrophilic polymer will then provide the balance.

In addition, the composite material may have a contact angle of less than 40° (i.e. a water contact angle) on a surface of the substrate coated with the block copolymer brush. In particular embodiments of the invention that may be mentioned herein, the composite material may have a contact angle of less than 20°, such as from 5 to 19°, on a surface of the substrate coated with the block copolymer brush. This shows that the composite material is hydrophilic on its surface.

In the context of the current invention, it will be appreciated that the terms “hydrophilic polymer” and “highly hydrophilic polymer” are intended to refer to materials that may display a contact angle as described above.

In the context of the current invention, the term “hydrophobic polymer” will be understood to mean a polymer that is more hydrophobic than the hydrophilic polymers used in the second block. For example, the hydrophobic polymer of the first block may have a contact angle that is greater than 40° (i.e. a water contact angle). It will be appreciated that the hydrophobic polymer must contain functional groups that can react to form a covalent bond to a moiety that contains a nitric oxide source.

Also, the composite material may provide a very smooth surface, which may be beneficial for use in certain applications (e.g. catheters) where the roughness of a surface may cause issues (e.g. abrasion and/or irritation to the tissues of a subject).

It is believed that one or more of these properties contribute to the advantages associated with the composite materials of the invention. For example, the use of a hydrophilic surface effectively inhibits biofilm formation by a broad spectrum of bacteria. The use of a hydrophilic and/or smooth surface also reduces or prevents immune cell activation, fouling by blood proteins or thrombus formation on the surface of the composite material.

The first block of the block copolymer may be formed from any suitable hydrophobic polymeric material or cationic polymeric material. For example, the first block may be formed from a polymer comprising one or more vinylogous monomers, where at least one of the one or more vinylogous monomers contains a functional group suitable to conjugate to a nitric oxide source. When used herein, the term “vinylogous” is intended to cover allylic monomeric materials as well. As noted above, the functional group suitable to conjugate to a nitric oxide source may be selected from one or more of the group consisting of an amine, a hydroxyl, a carboxylic acid, a carboxylic acid ester, and an epoxide. As will be appreciated, cationic polymers mentioned herein may contain ammonium species (i.e. positively charged amine groups), which groups are capable of conjugating to a nitric oxide source material, but are not necessarily used to do so, as certain composite materials disclosed here may only require the presence of cationic groups in the first block.

Examples of suitable vinylogous monomers include, but are not limited to acrylates, alkylacrylates (e.g. methacrylates), acrylamides, alkylacrylamides (e.g. methacrylamide), acrylic acids, alkyl acrylic acids, allylic monomer compounds and copolymers thereof. In particular embodiments that may be mentioned herein, the one or more vinylogous monomers may be selected from the group consisting of allyl amine, amino ethyl methacrylate (AEMA), hydroxyl ethyl methacrylate (HEMA), hydroxylethyl acrylate (HEA), hydroxyl ethyl acrylamide (HEAA), hydroxypropyl methacrylate (e.g. 2-hydroxypropyl methacrylate (HPMA)), hydroxybutyl acrylate, N-[Tris(hydroxymethyl)methyl] acrylamide, allyl alcohol, acrylic acid, methacrylic acid, glycidyl methacrylate (GMA), and ally glycidyl ether (AGE).

For example, the first block of the block copolymer may be a polyacrylate. In embodiments of the invention that may be mentioned herein, the first block of the block copolymer may be a polyacrylate. Examples of polyacrylates that may be mentioned herein include, but are not limited to, poly(hydroxyethyl methacrylate), poly(hydroxyethyl acrylate), poly(hydroxypropyl-methacrylate), poly(hydroxybutyl acrylate), and copolymers thereof.

The second block of the block copolymer may be formed from any suitable hydrophilic polymeric material. For example, the second block may be formed from a polymer comprising one or more vinylogous hydrophilic monomers. Examples of vinylogous hydrophilic monomers that may be mentioned herein include, but are not limited to a hydrophilic acrylate, a hydrophilic alkylacrylate (e.g. a hydrophilic methacrylate), a hydrophilic acrylamide, a hydrophilic alkylacrylamide (e.g. a hydrophilic methacrylamide), a hydrophilic acrylic acid, a hydrophilic alkyl acrylic acid, a hydrophilic allylic monomer compound, and copolymers thereof.

More particularly, the one or more vinylogous hydrophilic monomers may be selected from the group consisting of methyl terminated poly(ethylene glycol) methacrylate, methyl terminated poly(ethylene glycol) acrylate, allyl poly(ethylene glycol), oligoethyleneglycol methacrylate, a zwitterionic monomer (e.g. [2-(methacryloyloxy) ethyl] dimethyl-(3-sulfopropyl) ammonium hydroxide (SBMA), methacryloyloxyethyl phosphorylcholine (e.g. 2-methacryloyloxyethyl phosphorylcholine (MPC)), N-(3-methacryloylimino) propyl-N, N-dimethylammonio propanesulfonate (SBMAm), 3-(2′-vinyl-pyridinio) propanesulfonate (VPPS), carboxybetaine methacrylate (CBMA), 3-[(3-acrylamidopropyl) dimethylammonio] propanoate, sulfobetaine methacrylate, sulfobetaineacrylamide, 3-[(3-acrylamidopropyl) dimethylammonio] propanoate, and 3-[[2-(methacryloyloxy) ethyl] dimethylammonio] propionate), and a vinylogous-modified polysaccharide (e.g. dextran methacrylate, hyaluronate methacrylate).

For example, the second block of the block copolymer may be formed from methacryloyloxyethyl phosphorylcholine, sulfobetaine methacrylate, sulfobetaineacrylamide, 3-[(3-acrylamidopropyl) dimethylammonio] propanoate, 3-[[2-(methacryloyloxy) ethyl] dimethylammonio] propionate, and oligoethyleneglycol methacrylate, and copolymers thereof.

In particular embodiments that may be mentioned herein:
the first block may be formed from poly(hydroxyethyl methacrylate);
the second block may be formed from poly(sulfobetaine methacrylate); and
the nitric oxide source may be a tertiary S-nitrosothiol.

Also disclosed herein is a method for making a composite material as described above. Thus, in a further aspect of the invention, there is provided a method of forming a composite material as described hereinbefore, the method comprising the steps of:

(a) providing a substrate coated with a polymer brush, where the polymer brush is formed from a hydrophobic or cationic polymer covalently bonded to a surface of the substrate;

(b) reacting the hydrophobic or cationic polymer in the polymer brush with a hydrophilic monomer in a polymerisation reaction to form a di-block copolymer (block copolymer brush); and

(c) conjugating a nitric oxide source to the hydrophobic or cationic polymer of the block copolymer brush.

As will be appreciated the components mentioned in the method correspond to the materials discussed above (or can be deduced from said materials, such as the monomeric materials from the polymeric materials) and so will not be repeated here for the sake of brevity.

The substrate coated with a polymer brush will be understood to be an intermediate material that is used to form the final composite material described herein. Said intermediate material may be generated by analogy to the processes described in the examples below.

In an alternative aspect of the invention, there is disclosed a method for making a composite material that comprises a nitric oxide source conjugated to the hydrophobic or cationic polymer of the first block as described herein, said method comprising the steps of:

(a) providing a substrate coated with a block copolymer brush, where the block copolymeric brush is formed from:
a first block of a hydrophobic or cationic polymer covalently bonded to a surface of the substrate; and
a second block of a hydrophilic polymer, extending from the first block to form an outer surface of the block copolymer brush; and

(b) conjugating a nitric oxide source to the hydrophobic or cationic polymer of the first block.

The materials disclosed herein display potent broad-spectrum antibiofilm activity with excellent biocompatibility and antifouling properties. Given this, the composite material described herein may be used in the formation of articles that may be used in a number of applications. Thus, in a further aspect of the invention, there is provided an article formed from the composite material as described herein, wherein a substrate of the composite material has at least one surface that is coated with a block copolymer brush as described herein.

In a particular application that may be mentioned, the article may be a catheter formed from the composite material, where the catheter has an outer surface and an inner surface, and one or both of the outer surface and inner surface have been coated with a block copolymer brush as described herein.

In a further application that may be mentioned, the article may be a wound dressing formed from the composite material, where the wound dressing has an outer surface and an inner surface, and the inner surface is coated with a block copolymer brush as described herein.

It is noted that the articles may be formed from any of the composite materials disclosed herein. However, certain advantages may be tied to the use of composite materials that further comprise a nitric oxide source conjugated to the hydrophobic or cationic polymer of the first block.

Further aspects and embodiments of the invention will now be discussed with reference to the following non-limiting examples.

### EXAMPLES

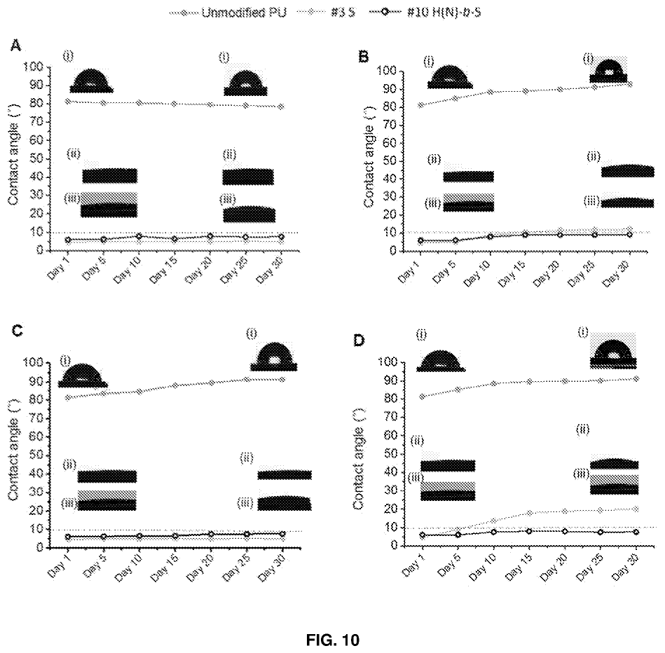

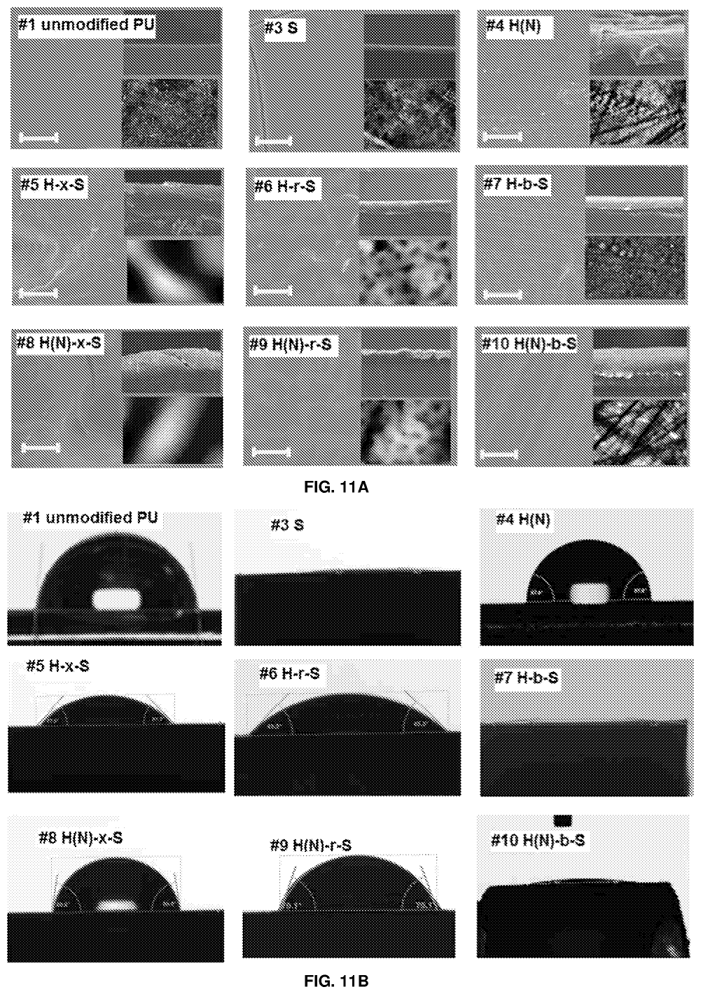

Disclosed here in Examples 1 to 11 is a composite material comprising a diblock copolymer H(N)-b-S (coating #10, Table 1) grafted from both the inner and outer surfaces of a slender polyurethane (PU) catheter via an ozone-initiated surface reversible addition-fragmentation chain-transfer (Ozone-surface-RAFT) block copolymerization (FIG. 10). The composite material is provided by first activating the PU surface with ozone to provide a high density of peroxide and/or hydroperoxide initiators on the surface. A subsurface (inner) block of poly(HEMA) (H) is then “grafted from” the surface initiators by polymerizing HEMA monomers in the presence of a chain transfer agent (CTA) and a thermal initiator (4,4′-azobis(4-cyanovaleric acid), ACVA). A subsequent RAFT-mediated block copolymerization of SBMA is then performed to produce the contiguous outer (surface) block (S). The resulting H-b-S diblock brush (coating #7, Table 1) is then post-modified by reacting the constituent H block with a RSNO which functions as a NO-donor (denoted as N) to provide the NO-emitting (#10) H(N)-b-S diblock brush (FIG. 10) as described in Example 2. The RSNO employed is 3-nitrosothiol-3-methylbutan-chloride (NTMB-Cl).

Materials and Methods

Polyurethane catheter (Micro-Renathane Tubing) with inner/outer diameter: 2.0 mm/3.5 mm was purchased from Braintree Scientific. 2-hydroxyethyl methacrylate (>99%) (HEMA), [2-(methacryloyloxy) ethyl] dimethyl-(3-sulfopropyl) ammonium hydroxide (sulfobetaine methacrylate, SBMA), triethylene glycol dimethacrylate (TEGDMA), ammonium iron(II) sulfate (Mohr's salt; (NH₄)₂Fe(SO₄)₂.6H₂O), 4-cyano-4-(phenylcarbonothioylthio) pentanoic acid (CPCPA, chain transfer agent), 4,4′-Azobis(4-cyanovaleric acid) (ACVA, thermal initiator), butylamine, oxalyl chloride, tert-butyl nitrite, 3-mercapto-3-methylbutan-1-ol, 3-(4,5-dimethylthiazol-2-yl)-2,5-diphenyltetrazolium bromide (MTT), fibrinogen, albumin and human serum were purchased from Sigma-Aldrich and used without further purification. Ozone was generated with an Arzocon RMU16-K3 using air as O₂ source. Polyurethane catheter (#1) control was used as received. Silver-coated catheter (#2) was purchased from a commercial supplier (confidential).

Bacteria: Methicillin-resistant Staphylococcus aureus (MRSA) BAA-38 and BAA-40, Vancomycin-Resistant Enterococci (VRE) V583, Methicillin-Resistant Staphylococcus epidermidis (MRSE) 35984, Pseudomonas aeruginosa PAO1, Acinetobacter baumannii 19606 and Klebsiella pneumoniae 13883 were obtained from ATCC. Escherichia coli UT189 was donated by another research group. Clinically relevant Multi-Drug Resistant (MDR) Gram-negative bacteria E. coli (ECOR-1), A. baumannii (AB-1), P. aeruginosa (PAER) and K. pneumoniae (KPNR-1) were obtained from Tan Tock Seng Hospital (TTSH), Singapore.

The surface functional groups of modified catheters were characterized using a Fourier-transform infrared spectrometer (FTIR) with ATR accessory at an incident angle of 90° (Nicolet 5700, Thermo Fisher Scientific, U.S.A). Water contact angles were measured with a drop shape analyzer, DSA-25, Kruss Scientific, Germany. Surface morphologies of the catheters were studied with Scanning Electron Microscopy (JEOL JSM-6701F, Japan) and Atomic Force Microscopy (Bruker Dimension Icon).

General Procedure 1: Ozone Treatment of Polyurethane (PU) Catheter

Unless otherwise specified, all catheters used were 5 mm in length with inner and outer diameters of 2.0 mm and 3.5 mm respectively. The surface coated catheters were all subjected to the ozone pre-treatment as described here. Polyurethane (PU) catheter was sectioned into 5 mm long pieces, which were cleaned with methanol followed by deionized water. Ambient air was used as input to the ozone generator and the output ozone was purified by passing it through a 15 cm sodium hydroxide column. The cleaned PU catheter pieces were put in a conical flask with ozone flowing into the flask at 0.6 L/min for 30 mins to introduce peroxide groups (step 1 of FIG. 1C). The ozone treated PU catheters were put under vacuum conditions (<10 Pa) at room temperature for 1 hr to remove oxygen/ozone diffused into the PU.

General Procedure 2: Synthesis of Nitric Oxide (NO) Release Precursor (NTMB-Cl)

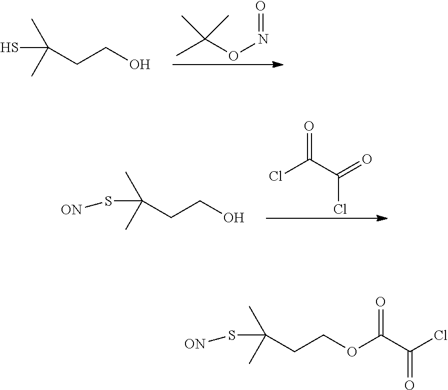

1.2 g of 3-mercapto-3-methylbutan-1-ol was dissolved in 10 mL cold ether and 1.03 g of tert-butyl nitrite was added dropwise. The mixture was reacted for 30 mins at 0° C. for full conversion of nitroso group. The reaction solution was then purified with partial vacuum to remove by-product and ether. The product, S-nitroso-3-mercapto-3-methylbutan-1-ol, was then quickly dissolved in 10 mL hexane with addition of 1.39 mL of triethylamine. This mixture was maintained at 0° C. 0.84 mL of oxalyl chloride was dissolved in 5 mL of hexane and added dropwise to the chilled S-nitroso-3-mercapto-3-methylbutan-1-ol solution. The mixture was reacted at 0° C. for 2 hrs to obtain S-nitroso-3-mercapto-3-methylbutan-1-Chloride (NTMB-Cl).

### Example 1: Coating of Diblock Copolymer on Ozone-Treated PU Catheter Via RAFT Polymerization ((#7) H-b-S)

Step 1: Grafting of First Block of Chain Transfer Agent Terminated Poly(hydroxyethyl methacrylate) Layer (poly(HEMA)) onto Catheter

A 10 wt % HEMA solution in 10 mL water and ethanol (4:1 v/v) mixture was prepared. 10 mg of thermal initiator 4,4′-Azobis(4-cyanovaleric acid) (ACVA) and 40 mg of chain transfer agent 4-cyano-4-(phenylcarbonothioylthio) pentanoic acid (CPCPA) were then dissolved in the solution. The solution was purged with Argon in a Schlenk tube for 30 mins. The ozone treated 5 mm long catheters (prepared following General Procedure 1) were then placed into the monomer solution, after which the solution was further purged for 5 mins and the tube was sealed. Surface ozone-RAFT polymerization was initiated by heating to 70° C. The polymerization reaction was carried out for 24 hrs and then quenched in an ice-bath. The coated PU catheters were washed and sonicated in pure ethanol to remove unattached homo poly(HEMA) and residual monomer.

Characterization of Coating Formed in Step 1:

FTIR absorption frequencies (cm⁻¹): 1742, 1020;

Thickness of coating as determined by SEM: 3 μm;

Measured root mean square height variation (RMS): 11.8±0.5 nm; and

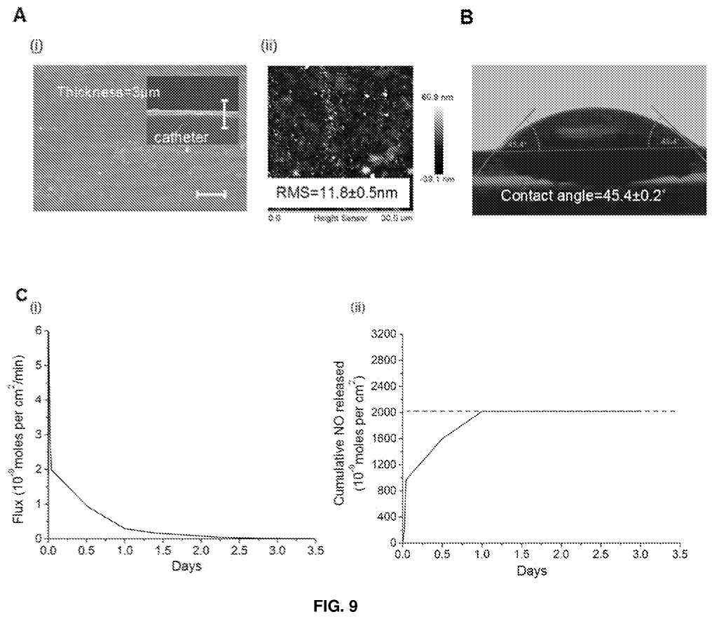

Contact angle: 45.4±0.2° (FIG. 9).

Additional thermal initiators (ACVA) were required even though peroxide groups were introduced onto the PU catheter surface (Langmuir 2005, 21, 450). It is believed that the additional thermal initiators act as scavengers for trace impurities which might terminate the polymerisation (Macromolecules 2002, 35, 610; Progress in Polymer Science 2011, 36, 845)

Step 2: RAFT Polymerization of Second Block of Poly(SBMA) to Form the Diblock Brush Covalently Grafted from Catheter (#7) H-b-S

A 10 wt % solution of SBMA in water and ethanol mixture (4:1 v/v) was prepared. 10 mg of thermal initiator ACVA was dissolved in this solution. The chain-transfer agent grafted catheter prepared in step (1) was placed in the solution. The solution was purged for 30 mins with Argon in a Schlenk tube. Polymerization was initiated by heating to 70° C. and carried out at this temperature for 12 hrs and then quenched in an ice-bath. The coated PU catheter was washed and sonicated in DI water to remove unattached homo poly(SBMA) and residual monomer. The terminal chain transfer agent was removed by heating the catheter in 10% butylamine in methanol solution at 40° C. for 1 hr. This produced the final coating (#7) H-b-S.

Characterization of Coating of (#7) H-b-S:

FTIR absorption frequencies: C═O ester at 1742 cm⁻¹, SO₃⁻ sulfonyl peak at 1040 cm⁻¹ and C—O—H peak at 1020 cm⁻¹;

Thickness of coating as determined by SEM: 8 μm;

Measured root mean square height variation (RMS): 7.9±0.1 nm; and

Contact angle: 5.4±0.1°.

It is believed that the chain transfer agent (CTA) in solution phase prevents radical-radical coupling of surface polymer brush, resulting in a more uniform and well-controlled second block (Macromolecules 2007, 40, 879) In other words, the CTA prevents undesired termination of the second block of the copolymer.

### Example 2: Attachment of Nitric Oxide Precursor onto (#7) H-b-S Coating to Form (#10) H(N)-b-S

Catheter (#7) H-b-S prepared in Example 1 was added into S-nitroso-3-mercapto-3-methylbutan-1-chloride (NTMB-Cl) solution (10 w/w % in hexane, 10 mL; prepared following General Procedure 2) and reacted with it for 12 hrs under ice-bath. The modified catheter was washed in methanol and water for 4 hrs at 0° C. to remove unreacted NTMB-Cl. This produced the final coating (#10) H(N)-b-S.

Characterization of Coating (#10) H(N)-b-S:

FTIR absorption frequencies: C═O ester at 1742 cm⁻¹ and SO₃⁻ sulfonyl peak at 1040 cm⁻¹;

Thickness of coating as determined by SEM: 10 μm;

Measured root mean square height variation (RMS): 15.4±3.4 nm; and

Contact angle: 8.0±0.4°.

### Example 3: Preparation of Reference Catheters

To evaluate the importance of the precision diblock copolymer structure, two controls which are (#1) uncoated catheter and (#2) silver-coated catheter, and six polymer coatings with different architectures (#3 to #6; #8 to #9) were prepared using the same monomers (2-hydroxyethyl methacrylate (HEMA) and sulfobetaine methacrylate (SBMA)) as summarised in Table 1.

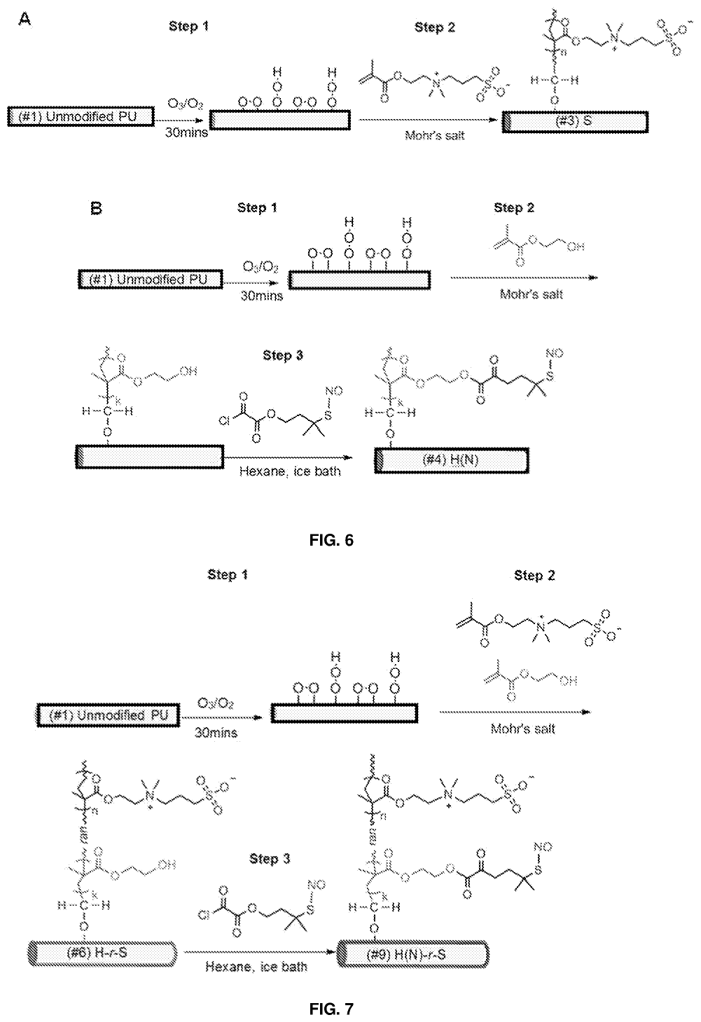

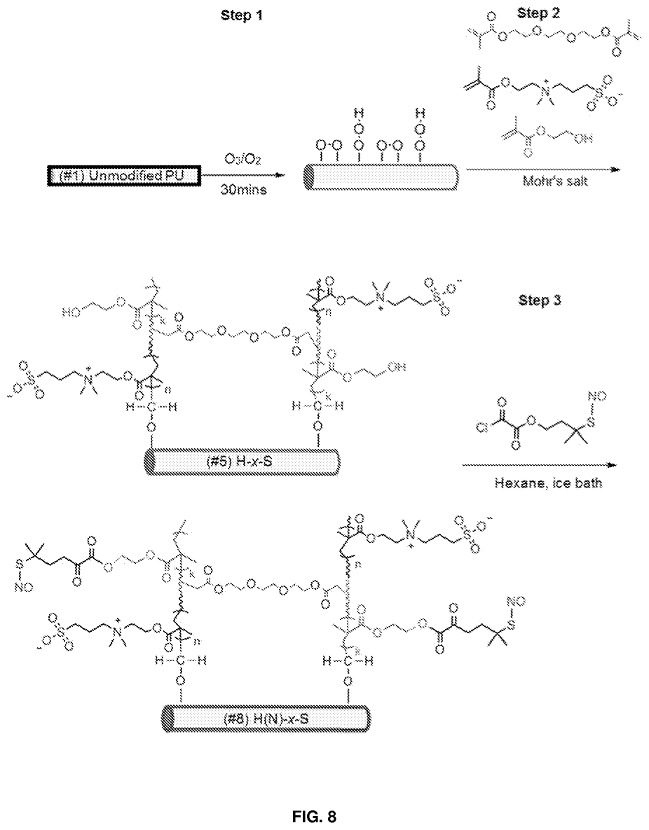

TABLE 1
Summary of coating polymers on polyurethane (PU) catheter

| Coating | | | | Synthetic |
|---|---|---|---|---|
| # | Label | Coating polymer | Method of synthesis | Scheme |
| 1 | Unmodified | None | Uncoated | |
| 2 | Silver | commercial silver | Purchased | |
| | | coated catheter | | |
| 3 | S | homo poly(SBMA) | Free radical | FIG. 6A |
| | | | polymerization of SBMA | |
| 4 | H(N) | homo poly(HEMA- | Free radical | FIG. 6B |
| | | NO) | polymerization of HEMA | |
| | | | and then reaction with | |
| | | | NO-donor (NTMB-CI) | |
| 5 | H-x-S | crosslinked hydrogel | Free radical | FIG. 8 |
| | | of poly(SBMA-co- | copolymerization of | |
| | | HEMA) | SBMA, HEMA with | |
| | | | crosslinker | |
| 6 | H-r-S | poly(HEMA-ran- | Free radical | FIG. 7 |
| | | SBMA) | copolymerization of | |
| | | | SBMA and HEMA | |
| 7 | H-b-S | poly(HEMA-block- | RAFT block | FIG. 1C |
| | | SBMA) | copolymerization of | |
| | | | HEMA followed by | |
| | | | SBMA | |
| 8 | H(N)-x-S | crosslinked hydrogel | Free radical | FIG. 8 |
| | | of poly(SBMA-co- | copolymerization of | |
| | | HEMA(NO)) | SBMA, HEMA with | |
| | | | crosslinker and then | |
| | | | reaction with NTMB-CI | |
| 9 | H(N)-r-S | poly((HEMA-NO)- | Free radical | FIG. 7 |
| | | ran-SBMA) | copolymerization of | |
| | | | SBMA and HEMA and | |
| | | | then reaction with | |
| | | | NTMB-CI | |
| 10 | H(N)-b-S | poly((HEMA-NO)- | RAFT block | FIG. 1C |
| | (optimum) | block-SBMA)) | copolymerization of | |
| | | | HEMA followed by | |
| | | | SBMA then reaction with | |
| | | | NTMB-CI | |

Coating of Homo Poly(SBMA) on Ozone-Treated PU Catheter ((#3) S)

A 10 wt % SBMA solution in 10 mL water and isopropanol (IPA) mixture (1:1 v/v)) was prepared. The monomer solution was purged in a Schlenk tube using Argon for 30 mins. The ozone treated 5 mm long catheter (prepared following General Procedure 1) was then placed into the monomer solution, after which the solution was further purged for 5 mins. Then, 8 mg of ammonium iron(II) sulphate was added to initiate the surface initiated free radical polymerization. The polymerization reaction was carried for 24 hrs at room temperature. The coated PU catheter was washed and sonicated in DI water to remove unreacted monomer and unattached homopolymer. This produced the final coating (#3) S.

Characterization of Coating (#3) S:

FTIR absorption frequencies: C═O ester at 1742 cm⁻¹ and SO₃⁻ sulfonyl peak at 1040 cm⁻¹;

Thickness of coating as determined by SEM: 3 μm;

Measured root mean square height variation (RMS): 4.8±0.5 nm; and

Contact angle: 4.7±0.1°.

Coating of Poly(HEMA) on Ozone-Treated PU Catheter and Attachment of Nitric Oxide Precursor ((#4) H(N))

A 5 wt % HEMA solution in 10 mL water and isopropanol (IPA) mixture (1:1 v/v)) was prepared. The monomer solution was purged in a Schlenk tube using Argon for 30 mins. The ozone treated 5 mm long catheter (prepared following General Procedure 1) was then placed into the monomer solution, after which the solution was further purged for 5 mins. Then, 8 mg of ammonium iron(II) sulphate was added to initiate the surface initiated free radical polymerization. The polymerization reaction was carried out for 24 hrs at room temperature. The polyHEMA-coated PU catheter was washed and sonicated in DI water to remove unreacted monomer and unattached homopolymer.

The poly(HEMA) coated catheter was then added into NTMB-Cl solution (10 w/w % in hexane; prepared following General Procedure 2) and reacted with it for 12 hrs under ice-bath. The modified catheter was washed in methanol and water for 4 hrs at 0° C. to remove unreacted NTMB-Cl. This produced the final coating (#4) H(N).

Characterization of Coating (#4) H(N):

FTIR absorption frequencies: C═O ester at 1742 cm⁻¹ and RSNO peak at 1160 cm⁻¹;

Thickness of coating as determined by SEM: 10 μm;

Measured root mean square height variation (RMS): 22.6±1.7 nm; and

Contact angle: 87.8±0.3°.

Coating of Cross-Linked Copolymer on Ozone-Treated PU Catheter Via Surface-Initiated Redox Polymerization ((#5) H-x-S), and Attachment of Nitric Oxide Precursor onto (#5) H-x-S Coating to Form ((#8) H(N)-x-S)

A solution containing 5 wt % SBMA, 5 wt % HEMA and 1 wt % TEGDMA in a 10 mL water and isopropanol (IPA) mixture (1:1 v/v)) was prepared. The solution was purged with Argon in a Schlenk tube for 30 mins. The ozone treated 5 mm long catheter (prepared following General Procedure 1) was then placed into the monomer/crosslinker solution and the solution was further purged for 5 mins. Then, 8 mg of ammonium iron(II) sulphate was added to initiate the surface initiated free radical polymerization. The polymerization reaction was carried out for 24 hrs at room temperature. The coated PU catheter was washed and sonicated in DI water to remove unreacted monomer and unattached homopolymer. This produced the final crosslinked hydrogel coating (#5) H-x-S.

Characterization of Coating (#5) H-x-S:

FTIR absorption frequencies: C═O ester at 1742 cm⁻¹, SO₃⁻ sulfonyl peak at 1040 cm⁻¹ and C—O—H peak at 1020 cm⁻¹;

Thickness of coating as determined by SEM: 10 μm;

Measured root mean square height variation (RMS): 200.5±8.7 nm; and

Contact angle: 45.5±0.2°.

The surface grafted catheter (#5) H-x-S was added into NTMB-Cl solution (10 w/w % in hexane; prepared following General Procedure 2) and reacted with it for 12 hrs under ice-bath. The modified catheter was washed in methanol and water for 4 hrs at 0° C. to remove unreacted NTMB-Cl. This produced the final coating (#8) H(N)-x-S.

Characterization of Coating (#8) H(N)-x-S:

FTIR absorption frequencies: C═O ester at 1742 cm⁻¹ and RSNO peak at 1160 cm⁻¹ and SO₃⁻ sulfonyl peak at 1040 cm⁻¹;

Thickness of coating as determined by SEM: 10 μm;

Measured root mean square height variation (RMS): 108.6±8.1 nm; and

Contact angle: 70.1±0.1°.

Coating of Random Copolymer on Ozone-Treated PU Catheter Via Surface-Initiated Redox Polymerization ((#6) H-r-S), and Attachment of Nitric Oxide Precursor onto (#6) H-r-S Coating to Form ((#9) H(N)-r-S)

A solution containing 5 wt % SBMA monomer and 5 wt % HEMA monomer in 10 mL water and isopropanol (IPA) mixture (1:1 v/v)) was prepared. The solution was purged with Argon in a Schlenk tube for 30 mins. The ozone treated 5 mm long catheter (prepared following General Procedure 1) was then placed into the monomer solution and the solution was further purged for 5 mins. Then, 8 mg of ammonium iron(II) sulphate was added to initiate the surface free radical polymerization. The polymerization reaction was carried out for 24 hrs at room temperature. The coated PU catheter was washed and sonicated in DI water to remove unreacted monomer and unattached homopolymer. This produced the final coating (#6) H-r-S.

Characterization of Coating (#6) H-r-S:

FTIR absorption frequencies: C═O ester at 1742 cm⁻¹, SO₃⁻ sulfonyl peak at 1040 cm⁻¹ and C—O—H peak at 1020 cm⁻¹;

Thickness of coating as determined by SEM: 7 μm;

Measured root mean square height variation (RMS): 108.9±4.3 nm; and

Contact angle: 47.3±0.3°.

The surface grafted catheter (#6) H-r-S was added into NTMB-Cl solution (10 w/w % in hexane; prepared following General Procedure 2) and reacted with it for 12 hrs under ice-bath. The modified catheter was washed in methanol and water for 4 hrs at 0° C. to remove unreacted NTMB-Cl. This produced the final coating (#9) H(N)-r-S.

Characterization of Coating (#9) H(N)-r-S:

FTIR absorption frequencies: C═O ester at 1742 cm⁻¹ and RSNO peak at 1160 cm⁻¹ and SO₃⁻ sulfonyl peak at 1040 cm⁻¹;

Thickness of coating as determined by SEM: 5 μm;

Measured root mean square height variation (RMS): 178.3±14.1 nm; and

Contact angle: 80.8±0.1°.

### Example 4: Preparation of 30 cm-Long PU Catheter Coated with #10 H(N)-b-S

30 cm PU catheter was cleaned using methanol and deionized water and subjected to the ozone treatment following General Procedure 1. The ozone treated PU catheters were put under vacuum conditions (<10 Pa) at room temperature for 1 hr to remove oxygen/ozone diffused into the PU. After activation, the catheter was connected to a peristaltic pump and put into a 3-neck flask (or plug-flow reactor) (FIG. 1B). The entire 30 cm catheter was submerged in the coating solution.

The surface block copolymerization reactions and RSNO attachment were carried out in the plug-flow reactor set-up following Examples 1 and 2 except that the volumes of polymerization and RSNO attachment solutions were 100 ml. The reaction conditions such as Argon purging, temperature, polymerization time and concentrations of the monomer, thermal initiator and chain transfer agent follow Example 1. The coating solution was circulated in the catheter at 10 mL/min. The surface grafting of NO release precursor onto longer (30 cm) PU catheter follows Example 2 except that the volume of NTMB-Cl solution was 15 mL.

Comparison of Surface Properties Between Unmodified PU Catheter (#1), Silver-Coated Catheter (#2) and Modified Catheters (#3 S-#10 H(N)-b-S)

Visual Appearance

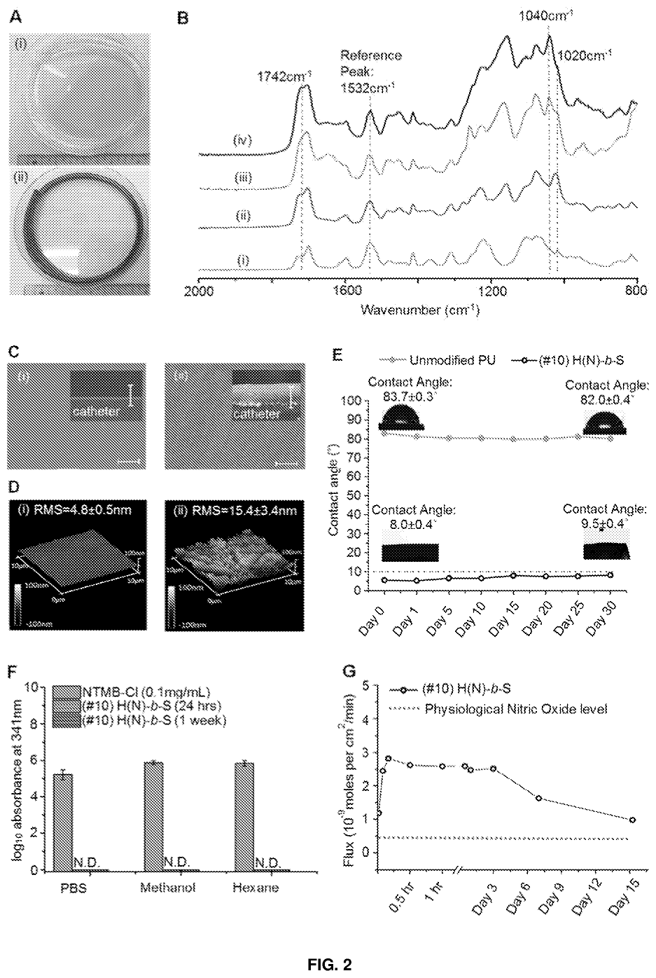

FIG. 2A(i) shows the gross visual appearance of a pristine 30 cm long PU catheter control to be transparent and almost colourless. After coating with (#10) H(N)-b-S (FIG. 2A(ii)), the colour of the catheter changes to a homogeneous dark green due to the presence of the tertiary nitrosothiol group in NTMB.

Attenuated Total Reflection-Fourier-Transform Infrared Spectroscopy (ATR-FTIR)

The chemical structures of the coatings were characterized using ATR-FTIR. Compared with the unmodified PU control (FIG. 2B(i)), the first block, poly(HEMA) (FIG. 2B(ii)), exhibited increased peak intensity at 1742 cm⁻¹ and a new peak at 1020 cm⁻¹, confirming the addition of an ester carbonyl group (C═O) and a hydroxyl (C—O—H) group due to HEMA.

The success of the second block copolymerization step to form the contiguous poly(SBMA) block of (#7) (H-b-S) was proved by the appearance of the sulfonyl signal at 1040 cm⁻¹ (FIG. 2B(iii)). The C—O—H group signal (1020 cm⁻¹) from the underlying poly(HEMA) block (H) was still detectable.

In the spectrum of (#10) H(N)-b-S (FIG. 2B(iv)), the disappearance of the poly(HEMA) brush C—O—H groups signal (at 1020 cm⁻¹) corroborates the successful reaction between C—O—H groups of (#7) and the chloride group of the NO-donor (NTMB-Cl). In addition, the ester carbonyl signal (1742 cm⁻¹) was further increased, corroborating the successful reaction of the chloride group of NTMB-Cl with hydroxyl groups of poly(HEMA).

The FTIR signals of the remaining coating (#3-#9) as described in the coating procedures corroborated their successful syntheses.

Scanning Electron Microscopy (SEM)

As shown by SEM images (FIG. 2C), the cross-section of the (#1) uncoated catheter was almost featureless but that of (#10) H(N)-b-S coated catheter showed a stratified coating with a total thickness of about 10 μm. The subsurface bottom layer was estimated to be 3-5 μm thick based on examination of the first poly(HEMA) block (FIG. 9).

3D Atomic Force Microscopy (AFM) Image and Measured Root Mean Square (RMS) Roughness

The AFM image of (#10) H(N)-b-S indicated that the block copolymer coating was very dense and thick with full coverage of the PU substrate (FIG. 2D). The (#10) H(N)-b-S coating was very smooth with measured root mean square (RMS) roughness of 15.4±3.4 nm. RMS of unmodified PU control is 4.8±0.5 nm.

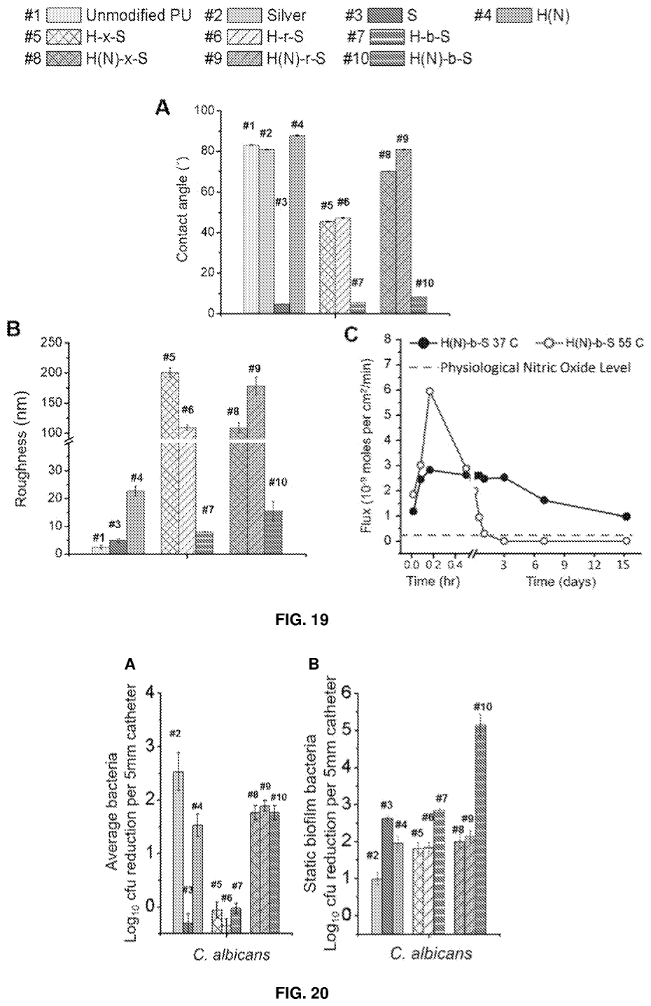

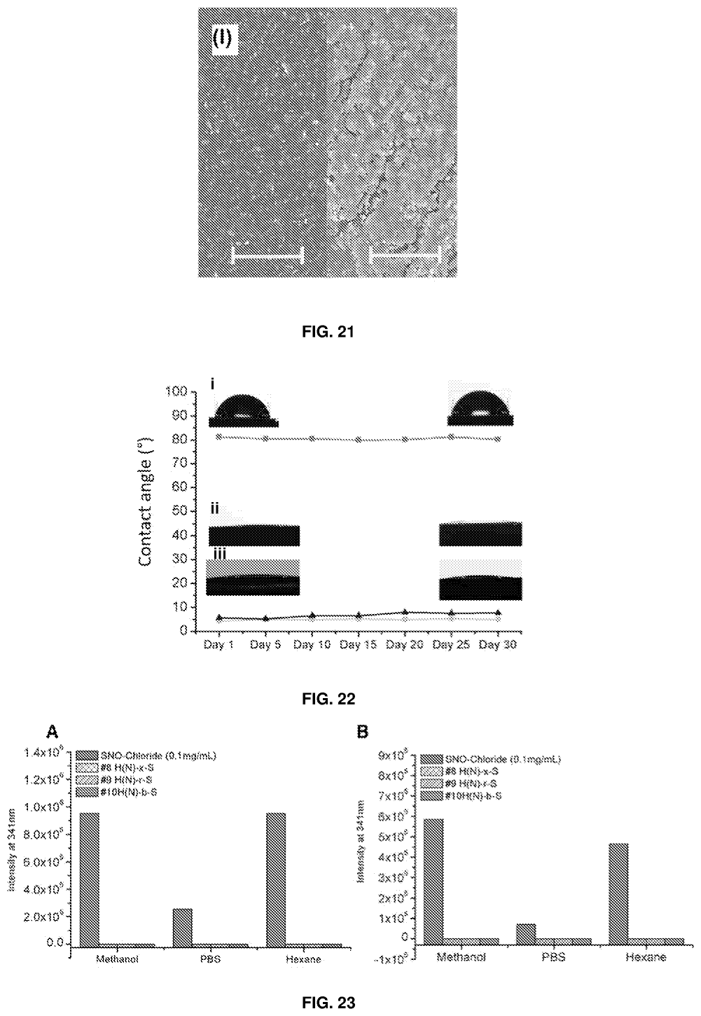

FIG. 19B shows that compared to the unmodified PU, #6 H-r-S and #5 H-x-S have much higher surface roughness (RMS >100 nm). The surface roughness of #7 H-b-S (RMS=7.9 nm) is not much larger than that of unmodified PU.

Water Contact Angle

The (#10) H(N)-b-S coating was superhydrophilic with superlow water contact angle (8.0±0.4°, FIG. 2E) compared to the unmodified PU control (83.7±0.3°, FIG. 2E).

In the initial studies, the modification process was tracked by water contact angle changes (FIG. 19C). The contact angle of the catheter declined from 82.3±0.1° (#1 Unmodified PU) to 45.4±0.1° (H) after grafting the first layer of polyHEMA. A single layer of polySBMA makes the surface even more hydrophilic, with contact angle reduced to 4.7±0.1° (#3 S; PU-S), but polySBMA does not have any functional groups for further modification. Grafting of the NO-release precursor to polyHEMA coating increases the contact angle to 87.8±0.1° (#4 H-N). The random (#6 H-r-S) and crosslinked (#5 H-x-S) coatings have contact angles of 47.3° and 45.4°, respectively, confirming that they are less hydrophilic than the block copolymer coating (#7 H-b-S) which has a contact angle of 5.4±0.1°, which is almost similar in hydrophilicity to the homo polySBMA coating (#3 S; 4.7±0.1°). After attachment of the NO-release precursor, the contact angles increase substantially to 80.8° (#8 H(N)-r-S) and 70.1° (#9 H(N)-x-S) respectively (FIG. 19A). Surprisingly, grafting of the NO-release precursor to the block copolymer only slightly increases the water contact angle to 8.0° (#10 H(N)-b-S), which indicates that the intrinsic hydrophilicity of the surface zwitterionic polymer layer is preserved.

Effect of Long-Term Incubation in Water and Media on Contact Angles

The (#10) H(N)-b-S brush coating was also chemically stable as shown by the very small increase in contact angle)(<10.0° when incubated in water (FIG. 2E) and various media (PBS, serum, and bacterial inoculum, FIG. 10) for 1 month. This indicates long term stability of the hydrophilic properties.

Quantification of Surface Peroxide on Polyurethane Catheter

The concentration of surface peroxide groups was calculating by titration against sodium thiosulfate following Keiji's protocol (Polymer Chemistry 1993, 31 (4), 1035). 25 mL IPA was added to the ozone treated catheter, followed by 1 mL saturated KI solution with 1 mL acetic acid. The mixture was heated and kept at boiling point for 5 mins with constant stirring by a magnetic bar and titrated with 0.01 mM Na₂S₂O₃ until the yellow colour completely change to white. The calculation procedure of surface peroxide group density is summarized below.

Volume of Sodium thiosulfate solution (0.01 mM) used: 3.30 mL Moles of peroxide=moles of thiosulfate titrated:

0.01×10⁻³×3.30×10⁻³=3.3×10⁻⁸ mole of peroxide per 5 mm of catheter

Calculation of Surface Peroxide Group Density:

$$σ = \frac{No . of peroxide group on 5 mm of cateter}{Surface area of 5 mm cathter}$$

$$σ = \frac{3.3 \times 1 {0}^{- 8} \times 6.023 \times 1 {0}^{23}}{\begin{matrix}0.25 \times 0.5 \times 3.14 + 0.4 \times 0.5 \times 3.14 + \\ 314 ( {( 0.5 \times 0.4 )}^{2} - {( 0.5 \times 0.25 )}^{2} ) \times 2\end{matrix}}$$

$$σ = 169 / {nm}^{2}$$

The calculations indicate that there are 169 peroxide initiators/nm².

NO Donor Leaching and NO Release Profile

Measurement of NO Release Precursor Leaching from Modified Catheter

To assess the leachability of the grafted NO-donor, (#10) H(N)-b-S coated catheter was extracted with PBS solution, polar (methanol) and nonpolar (hexane) solvents and analyzed after 24 hrs and 1 week (FIG. 2F). Specifically, the measurement of NO release precursor leaching from modified catheter followed ISO 10993-1 protocol. 5 mm pieces of modified catheters were extracted using PBS, methanol and hexane. The extractants were analysed using HPLC (Shimadzu LD-20) with UV detector (Shimadzu SPD-20) measuring absorbance at 341 nm (0.1 mg/mL of the NO release precursor, NTMB-Cl was used as standard).

No leaching of the NO-donor was detected in the 341 nm absorbance measurements (FIG. 2F). These non-leaching results prove the long-term stability of the grafted polymer coating (#10). The RSNO donor is also not leachable from either the random (#8) or the crosslinked (#9) coatings.

Measurement of NO Release Profile from Modified Catheter ((#10) H(N)-b-S)

The rate of release of NO from the catheter varies with temperature. To quantify this initially, 0.5 cm catheter samples were soaked in 1 mL PBS and incubated at specific temperatures.

Acute and long-term release of NO was measured using Apollo 1000 NO detector from World Precision Instruments. In an optimized procedure, 5 mm catheter samples were incubated in 1 mL of 10 mM PBS at pH 7.5 with 100 μM EDTA in darkness at 37° C. Real-time release of NO into the solution was measured using a TBR 1025 free radical analyser with Nitric Oxide probe from World Precision Instruments.

For estimating the total RSNO concentration after grafting onto the polymer, the RSNO-catheter was heated to 55° C. and the NO-release flux was tracked until NO release was completed. The amount of grafted RSNO was calculated from the area under the NO-release flux curve.

Using the Apollo 1000 NO detector with the catheter immersed in PBS at 37° C. (FIG. 19E), NO flux reaches the maximum (2.45×10⁻⁹ mol/cm²/min) in 10 mins and remains at that level for 72 hours (3 days), after which it declines slightly but is still higher than the natural NO flux from epithelial cells (0.5×10⁻¹⁰ mol/cm²/min). At higher temperature (55° C.), the early-time release is faster, which exhausts the NO-release precursor within 2-3 days (FIG. 19C)

Using the optimized procedure, NO release at 37° C. (FIG. 2G) can be maintained over 15 days (375 hours) at a higher level than the endogenous NO flux from epithelial cells. The density of NO-release precursors grafted on catheter is estimated to be 2.02×10⁻⁶ mole per cm² (FIG. 9C).

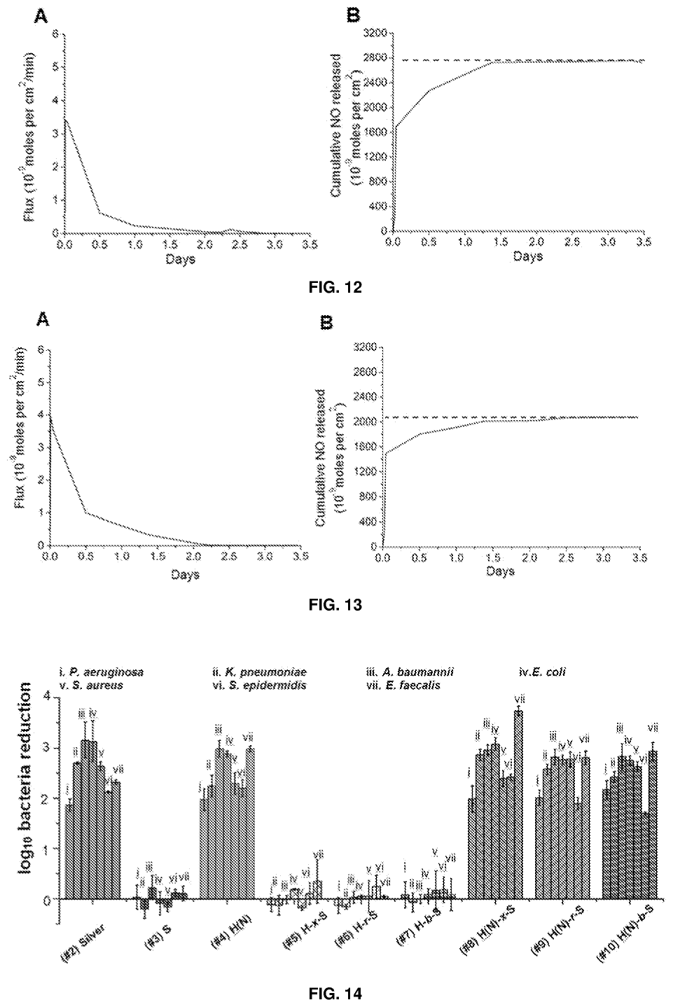

The RSNO donor grafting density for crosslinked (#8) and random (#9) coatings are estimated to be 2.75×10⁻⁶ mole per cm² (FIG. 12) and 2.03×10⁻⁶ mole per cm² (FIG. 13) respectively.

### Example 5: Antimicrobial and Antibiofilm Efficacy of Modified Catheters

General procedure for Bacteria inoculum preparation: microbe suspensions were prepared by scratching colonies from culture plates and dipped in protein rich medium (Tryptic Soy Broth, TSB for bacteria and yeast mold broth for fungus). The inoculum was incubated with shaking at 225 rpm at 37° C. (28° C. for fungus) to obtain the overnight culture.

General procedure for preparation of catheters for testing: catheter samples including control sample were sterilized with 75% ethanol for 10 minutes at room temperature, and then soaked in phosphate buffer (PBS) overnight at 4° C. before testing.

Acute (2 hrs) surface antimicrobial assay: The bacteria or fungus overnight cultures were washed thrice with PBS and resuspended in PBS at 10⁸ cfu/mL. 10 μL of the suspension was spread onto each catheter piece. The catheter pieces were then incubated at 37° C. for 2 hours with humidity not less than 90%. After the incubation, the catheter pieces were washed thoroughly using 100 μL of PBS, to recover the microbes. CFU numbers were counted by serial dilution of the recovered bacteria suspensions and culture on Lysogeny Broth Agar plates (Yeast Malt (YM) Agar for fungus). All tests were performed one time with 3 independent biological replicates.

24 hrs Static Biofilm Growth Test: Catheter pieces were immersed in 1 mL TSB in a 24-well plate (YM medium for fungus). 10⁶ CFU/mL microbe suspension in 10 mM phosphate buffer saline (PBS, pH 7.4) was made by diluting the overnight inoculum. 8 μL of the PBS suspension of bacteria suspension was added to each well. The 24-well plate was incubated at 37° C. for 24 hours with orbital shaking at 110 rpm. Catheter samples were then removed and washed gently thrice with PBS buffer to remove unattached or planktonic bacteria. Each catheter sample was then placed in an Eppendorf tube with 1 mL PBS buffer. The tube was placed in an ultra-sonicator bath for 15 minutes at 0° C. and vortexed for 5 minutes to strip and remove the attached biofilm from the catheter surface. The microbial suspension was serial diluted in PBS buffer, then grown on Lysogeny Broth Agar plates (YM Agar for fungus) at 37° C. overnight followed by CFU counting. All tests were performed one time with 3 independent biological replicates.

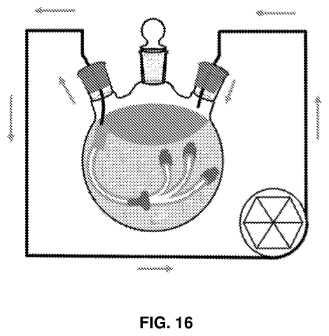

Long term Intraluminal Biofilm Growth Test (For 30 cm PU catheter): The antibiofilm efficacy of longer (30 cm) catheter was tested using an in-vitro model of exposure to physiological fluids (FIG. 16). That is, a circulatory setup of physiological fluids. The catheters were first incubated in human serum for 30 min to simulate contact with blood. The catheter samples were then incubated in PBS suspension of bacteria (MRSA BAA-38 or P. aeruginosa) at 10⁸ CFU/mL for 2 hours for initial attachment of bacteria which would form intraluminal biofilms. The catheter samples were then attached to a peristaltic pump which circulated Brain Heart Infusion (BHI) medium maintained at 37° C. at 0.7 mL/min for defined study periods for biofilm growth. After the biofilm growth periods, the catheter samples were removed from the test apparatus and 3 pieces of 5 mm long catheters were cut from the initially 30 cm long catheter. The catheter pieces were washed gently thrice with PBS buffer to remove unattached bacteria. Each catheter sample was then placed in an Eppendorf tube with 1 mL PBS buffer. The tube was placed in an ultra-sonicator bath for 15 minutes at 0° C. and vortexed for 5 minutes to strip and remove the attached biofilm from the surface. The microbial suspension was serial diluted in PBS buffer, then grown on Lysogeny Broth Agar (LB Agar) plates (YM Agar for fungus) at 37° C. overnight followed by CFU counting. All tests were performed one time with 3 independent biological replicates.

For all in vitro catheter antibiofilm efficacy tests, the log reduction in microbe numbers of modified catheters compared with (#1) unmodified control catheter was calculated as:

$$Log Reduction = {log}_{10} ( \frac{CFU of control}{CFU of coated catheter} )$$

Results

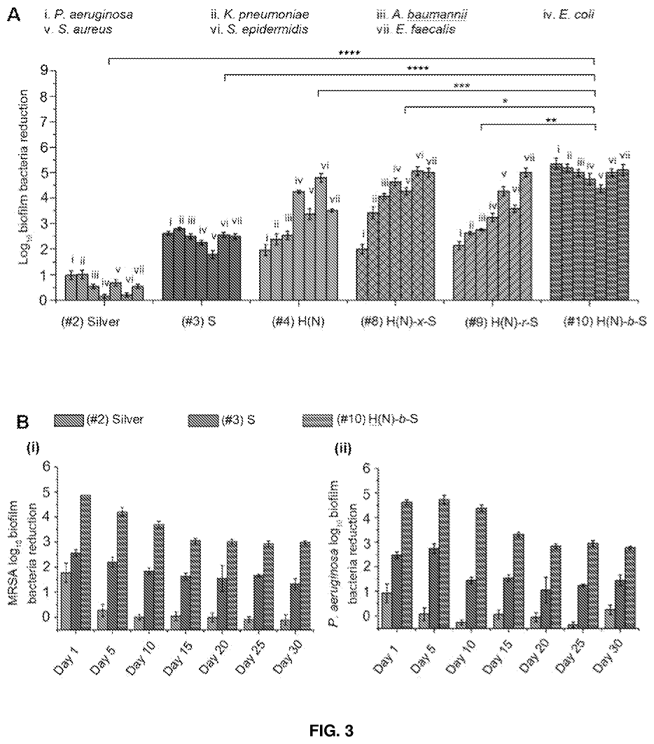

The purely hydrophilic coatings (#3S, #5H-x-S, #6H-r-S and #7H-b-S) show no bactericidal efficacy (FIG. 3 and FIG. 14). The bacteria were loaded directly on the catheter surface for 2 h before counting. Only the NO-emitting coatings, whether homopolymer- or copolymer-derived (#4H(N), #8H(N)-x-S, #9H(N)-r-S and #10H(N)-b-S), show significant antibacterial effect (≥2 log₁₀, ≥99.0%) against all pathogens tested, specifically Gram-negative and Gram-positive bacteria and a fungus (specifically E. coli UT189, K. pneumoniae ATCC 13883, P. aeruginosa PAO1, A. baumannii ATCC 19606, methicillin-resistant Staphylococcus aureus (MRSA) BAA40, vancomycin-resistant Enterococci (VRE) V583, methicillin-resistant Staphylococcus epidermidis (MRSE) ATCC 35984; and Candida albicans 90028). The antibacterial efficacies of these NO-emitting catheters (#4, #8-#10) were comparable to the commercial silver-coated catheter (#2 Silver). The structure of the copolymer coating (i.e., random, cross-linked or block, #8-#10) had little effect on the bactericidal and fungicidal efficacy (FIG. 20A).

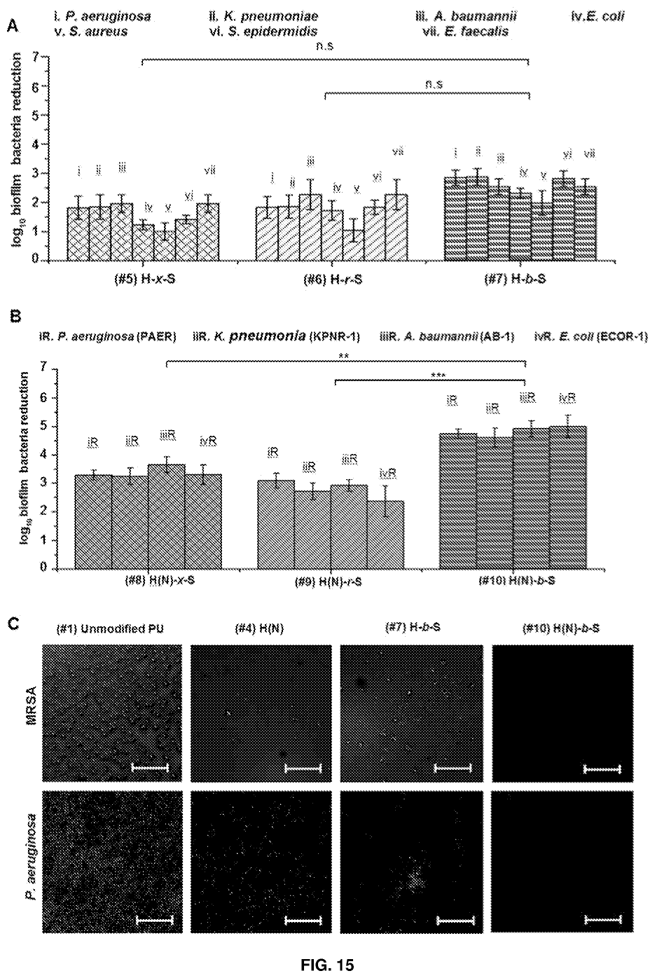

With respect to the short-term (24 hrs) antibiofilm efficacy of the coatings, only the diblock copolymer brush (#10) with NO-release functionality and a hydrophilic surface layer provided >4.0 log₁₀ inhibition against both Gram-positive and Gram-negative (FIG. 3A(i) bacteria tested. Comparing the purely hydrophilic coatings (#3, #5-7), (#3) and (#7) brushes with surface poly(SBMA) layer perform slightly better than (#5) and (#6) by 0.5-1.5 log₁₀ orders of magnitude against both Gram-positive and Gram-negative bacteria (FIG. 15A). Comparing the NO-releasing coatings (#4, #8-#10), they were all effective against all the Gram-positive species tested (FIG. 3A(i)). However, against the entire panel of Gram-negative bacteria tested (P. aeruginosa, K. pneumoniae, A. baumannii and E. cob), (#10) was the only NO-emitting coating that achieved 4.7-5.3 log₁₀ inhibition. The (#9) NO-emitting random copolymer has generally poor (2.1 to 2.8 log₁₀) inhibition against the Gram-negative bacteria except E. coli (with 3.2 log₁₀ reduction). The (#8) NO-emitting hydrogel has poor to moderate (2.0 and 3.4 log₁₀) reductions of P. aeruginosa and K. pneumoniae which were about 2.0 log₁₀ less than (#10); it has good (4.1 and 4.6 log₁₀) reduction against A. baumannii and E. coll. With Candida albicans, only the diblock copolymer brush with NO release (#10) provided significantly high antibiofilm effect of >4.5 log₁₀ (FIG. 20B)

The in vitro efficacy of #8-#10 against four multi-drug resistant (MDR) strains was tested before the in vivo studies. The NO-release block copolymer coating (#10) shows 4.7-5.0 log₁₀ inhibition of biofilm formation against various MDR Gram-negative bacteria, which is 93% to 98% more potent than the crosslinked NO-releasing coating (#8), and 97-99.7% more potent than the random NO-releasing coating (#9) (FIG. 15B).

MRSA and P. aeruginosa bacteria adhesion on various catheters were also observed with fluorescence microscopy (FIG. 15C). On the (#10) coated catheter surface, neither MRSA or P. aeruginosa was found. In contrast, on (#1) unmodified, (#4) purely NO-emitting and (#7) purely hydrophilic coatings, both MRSA and P. aeruginosa bacteria were found to various extents. Hence, of the various NO-emitting formulations, only (#10) H(N)-b-S with an antifouling surface layer and a hidden subsurface NO-donor layer achieves excellent (4.4-5.3 log₁₀) antibiofilm effect against both Gram-positive and Gram-negative bacteria. The (#10) H(N)-b-S coating is the most effective in terms of antibacterial and antibiofilm effect for the broad spectrum of bacteria tested.

The in vitro long-term (30 days) antibiofilm properties of 30 cm long (#10) coated catheter were evaluated and compared with a few controls, i.e. (#1) uncoated, (#2) silver-coated and (#3) poly(SBMA)-coated catheters, using a home-made intraluminal circulatory setup (FIG. 16) with a biofilm-promoting medium. For both MRSA and P. aeruginosa (FIG. 3B(i) and (ii), the (#2) silver-coated catheter showed limited antibiofilm efficacy (˜90% inhibition) at Day 1 but developed biofilm from Day 5 onwards. The purely hydrophilic coating (#3) showed moderate 30-day antibiofilm effect with 1.6 and 1.5 log₁₀ inhibitions for MRSA and P. aeruginosa respectively. The (#10) H(N)-b-S coating retained about 99.9% inhibition against both MRSA (3.1 log₁₀ inhibition) and P. aeruginosa (3.0 log₁₀ inhibition) after 30 days. This study proved the excellent in vitro long-term (30 days) efficacy of (#10) H(N)-b-S and is the first report of a catheter coating with such high (around 3.0 log₁₀) long-term (30 days) antibiofilm effect against both Gram-positive and Gram-negative bacteria.

### Example 6: Cytotoxicity of Extracts of Modified Catheters on Mammalian Cell Lines

Cytotoxicity Assay

The cytotoxicity assay employed an extraction model adapted from ISO 10993-5 protocol. 3T3 fibroblasts, Human Embryonic Kidney 293 cells (HEK), Human Dermal Fibroblasts (HDF) and HepG2 cell lines obtained from ATCC were cultured in DMEM medium supplemented with 10% foetal bovine serum (FBS) and antibiotics (glutamine (2 mM), penicillin (100 units/mL), and streptomycin (100 μg/mL)). The cells were maintained at 37° C. in a humidified incubator with 5% CO₂ until a monolayer, with greater than 80% confluence, was obtained.

Catheter pieces (5 mm long) were sterilized by soaking in ethanol/water (75/25) in 10 minutes at room temperature and then soaked in PBS overnight at 4° C. Sterilized catheter pieces of 5 mm length were incubated in DMEM complete medium in sterile cell culture plates. The plates were incubated at 37° C. with 5% CO₂ in a humidified incubator for 24 hrs to extract soluble material from the catheter.

After 24 hrs-extraction, the cell line culture medium was discarded from the culture plates, and the cells were rinsed with PBS and the catheter extractants were added into the plate wells. The plates were then incubated for 24 hrs at 37° C. with 5% CO₂ in a humidified incubator. Then, 3-(4,5-dimethylthiazol-2-yl)-2,5-diphenyltetrazolium bromide, (MTT) (200 μL, 5 mg/mL) was added into each well and the plates were incubated under the same condition for 4 hrs. The MTT was aspirated and dimethyl sulfoxide (DMSO) was added into each well and the plates shaken (150 rpm) for 15 mins. The absorbance of each well was measured at 570 nm using a microplate reader spectrophotometer (BIO-RAD, Benchmark Plus). All the assays were performed 3 times with 3 independent biological replicates. Cell viability was calculated by the formula:

$$\% Cell viability = \frac{Absorbance from cells incubated with catheter extractant}{Absorbance from control cells incubated in pure DMEM} \times 100 \%$$

Results

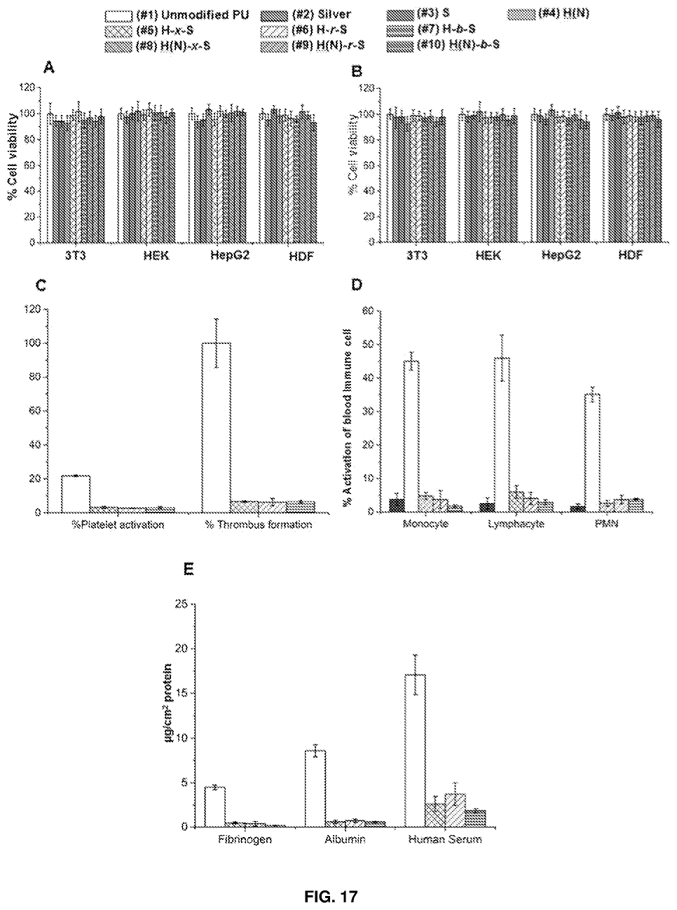

All control and coated catheters (#1 to #10) showed no toxicities toward all tested mammalian cell lines: 3T3 fibroblasts, human embryonic kidney (HEK 293) cells, human liver hepatoma (HepG2) cells and human dermal fibroblasts (FIG. 17A). Extractants from all catheter coatings, including the NO-emitting coated catheters (#4, #8-#10) show >80% cell viability for all cell lines, indicating good biocompatibility of the coating.

### Example 7: Surface Thrombus Formation Resulting from Modified Catheters

Catheter-triggered platelet activation and thrombus formation were measured by in vitro incubation of the catheter with whole rabbit blood. The activated platelets were labelled with antibodies and tracked by Fluorescence-Activated Cell Sorting (FACS) analysis. Thrombus formation on the surface was quantified by measuring Adenosine 5′-diphosphate (ADP) trapped in thrombi using 4-nitrophenyl phosphate disodium salt hexahydrate.

Thrombus Activation and Formation Test

Whole rabbit blood was freshly collected from the jugular vein of female New Zealand white rabbits provided by ASTAR and used immediately.

Sterilized 5 mm catheters were incubated with 100 μL of whole rabbit blood for 2 hrs in a 96-well plate. The catheters were then washed with PBS thrice to remove unattached protein or blood cells. For quantification of thrombus formation by platelet aggregation test using adenosine diphosphate (ADP) assay: the catheter pieces were incubated with 100 μL of 5% disodium 4-nitrophenyl phosphate for 2 hrs. The catheters were removed and 100 μL of 1M sodium hydroxide was added to the solution. The intensity of each well was measured with a plate reader under excitation wavelength of 405 nm. The test was performed one time with 3 independent biological replicates.

For Scanning Electronic Microscopy observation of surface thrombi on catheter pieces, the blood incubated catheter pieces were washed with PBS thrice to remove unattached protein or blood cells and fixed with 4% paraformaldehyde in PBS solution overnight at 4° C. The fixed catheter pieces were dehydrated in a graded ethanol series (25%, 50%, 75% and 100%). After dehydration, the ethanol was removed with an Argon gas flow. The dried samples were imaged with a Scanning Electron Microscope (JEOL JSM-6701F, Japan).

Results

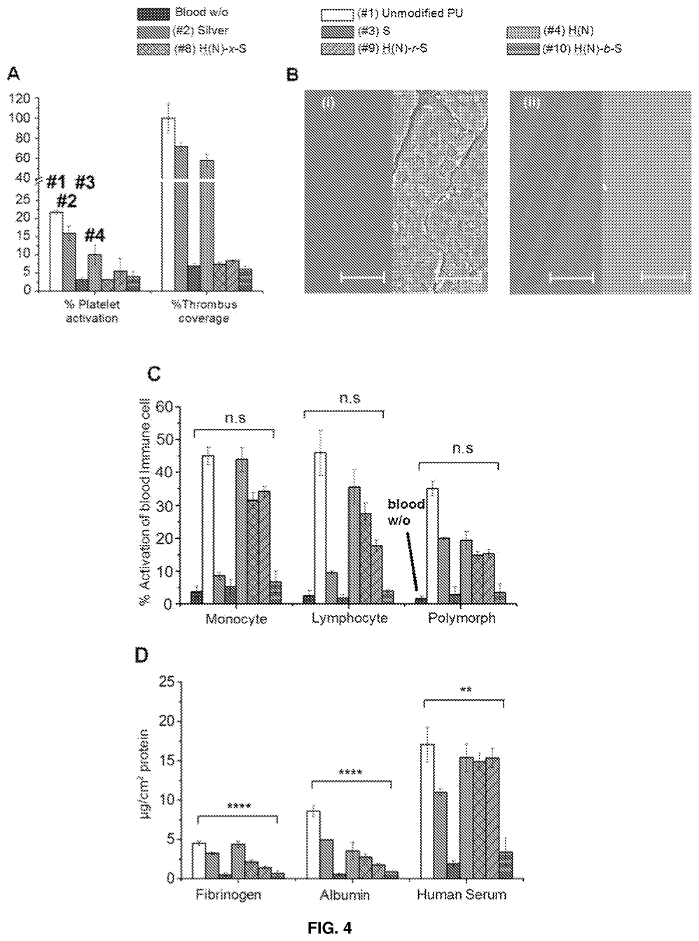

Upon incubation with rabbit whole blood for 2 hrs, the (#1) unmodified catheter (FIG. 4A) produced 22.5% activation of platelets and was densely covered with thrombi (FIG. 4B(i)).

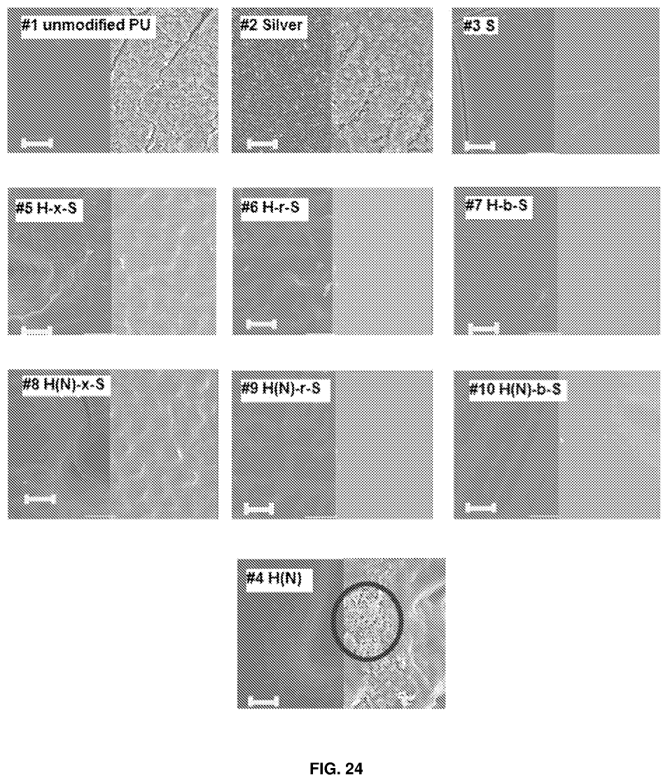

This level was set as the 100% thrombus formation control mark for comparison with other coatings. The (#2) silver-coated catheter showed 15.1% platelet activation and 73.2% thrombus formation. The purely NO-emitting coating ((#4 H(N))) (FIG. 4A) showed moderate platelet activation (˜10%) and significant (58.6%) thrombus formation. This is confirmed by SEM (FIG. 24). It is believed thrombus formation was due to the hydrophobic coating (contact angle of 87.8) as #4 H(N) lacks the zwitterionic component (S). The purely hydrophilic and the hydrophilic NO-coated catheters ((#3, #5-#10, FIG. 4A and FIG. 17B) showed low platelet activation (<10% activation) and low thrombus formation (<10%).

As confirmed by SEM in FIG. 4B(ii), (#10) H(N)-b-S coated catheter showed a clean surface with no thrombus formation. High coverage with thrombi was seen on unmodified catheter (#1, FIG. 4B(i) and FIG. 24). No thrombi were found for the random copolymer or the cross-linked copolymer with/without NO (Coating #5-#6, #8-#9, FIG. 24).

### Example 8: Immunological Response from Blood Immune Cells Towards Modified Catheters

Whole rabbit blood was used to test blood immunological response triggered by the catheters. Whole rabbit blood was freshly collected from the jugular vein of female New Zealand white rabbits by ASTAR and used immediately. Sterilized 5 mm catheters were incubated with 100 μL of the rabbit whole blood for 30 mins. The blood was then fixed with 4% paraformaldehyde for 10 mins at room temperature. The excess paraformaldehyde was then removed, and the cells were washed twice with PBS. Specific antibodies were dissolved in PBS and incubated with the blood samples for 1 hr (lymphocytes: CD3-APC and CD25-FTIC; monocytes: CD14-APC, CD11b-FTIC; platelets: CD41-FTIC, CD62p-APC; Polymorphs: CD66b-APC, CD11-FTIC). The labelled blood cells were then washed twice with PBS to remove excess antibodies. Cell counts were performed using Attune NxT Acoustic Focusing Cytometer. Blood without any treatment was used as negative control. The tests were performed one time with 3 independent biological replicates.

Results

The (#1) unmodified PU catheter (FIG. 4C) triggered significant activation of immune cells (i.e. monocytes (45%), lymphocytes (48%), and polymorphs (34%)) as measured by FACS. The (#2) silver-coated catheter (FIG. 4C) also triggered a substantial degree of activation of monocytes (8.2%), lymphocytes (9.5%) and polymorphs (20.0%). The purely hydrophilic coatings (#3, #5-#7) triggered low levels (2-6%) of immune cell activation which were only slightly higher than the endogenous level (1-4%, FIG. 4C, FIG. 17D) of blood immune cell activation in the absence of a trigger (negative control, i.e. blood without treatment). Interesting, the NO-donor modified random copolymer (#8) and crosslinked hydrogel (#9) show significant levels (20-30%) of activation of immune cells (FIG. 4C) compared with their parent non-NO emissive purely hydrophilic coatings (#5, #6). On the other hand, the NO-emitting diblock copolymer brush coating (#10) (FIG. 4C) showed low activation levels for lymphocytes (6.4%), monocytes (3.9%) and polymorphs (3.7%) which was comparable to the endogenous levels. Without wishing to be bound by theory, it is believed that the hydrophobic NO-donor which is provided in the subsurface poly(HEMA) avoids contact with immune cells and reduces their activation compared with alternative coating structures.

### Example 9: Surface Antifouling Test Using Blood Protein and Human Serum

The protein fouling resistances of the coatings was evaluated by incubating the catheters with 1% fibrinogen, 10% albumin or 50% human serum which contains a complex mixture of blood proteins.

Catheter pieces (5 mm long) were sterilized in 75% ethanol and soaked in PBS overnight before testing. The catheters were incubated with 1% fibrinogen, 10% albumin or 50% human serum at 37° C. for 24 hrs. The catheters were then removed and washed thrice with PBS. The washed catheter samples were put in 1 mL of 1% SDS, shaken at 110 rpm for 2 hrs and sonicated for 10 mins. The catheters were removed and the SDS solutions with protein detached from the catheters were incubated with bicinchoninic acid (BCA) protein assay kit solution for 30 mins at 60° C. The OD values of the SDS solutions were then measured at 562 nm. The protein concentrations were calculated according to the calibration curve provided in the kit.

Results

The hydrophobic coatings ((#1) unmodified, (#2) silver coated- and (#4) H(N)-coated catheters) all exhibited high levels of protein fouling (FIG. 4D; their contact angles: 83.7°, 80.8° and 87.8° respectively) (FIG. 2E). These results mirror those shown in the immune cell activation test. The purely hydrophilic coatings (#3, #5-#7) exhibited reduced fouling of <0.5 μg/cm² for fibrinogen, <1 μg/cm² for albumin and <4 μg/cm² for human serum (FIG. 17E). For catheters functionalized with NO-donor, only (#10) (and not #8 and #9) exhibited low protein fouling. The hydrophilic poly(SBMA) surface layer in (#10) H(N)-b-S coated catheter resists protein fouling to achieve comparable protein resistance to non-NO-releasing (#7) H-b-S coating (FIG. 4D).

### Example 10: In Vivo Murine Subcutaneous Catheter Implantation Model (Infection and Immunity 2003, 71 (2), 882)

The experiments were performed according to the Institutional Animal Care and Usage Committee (IACUC) of Nanyang Technological University (approved protocol number IACUC A18051, with approved amendment for subcutaneous catheter implantation).

In an initial study, overnight cultured bacteria were diluted in 1% TSB in PBS to give a final concentration of about 10⁷ CFU/mL. Catheters #10, #2, #3 and #7 were incubated with bacteria inoculum at 37° C. for 2 hrs. In the final study, overnight cultured bacteria were diluted in 1% TSB in PBS to give a final concentration of about 10⁶ CFU/mL and catheters #8, #9 and #10 were incubated with bacteria inoculum at 37° C. for 2 hrs.

7 to 9 weeks old female BALB/c mice were anesthetized by injection of Ketamine and shaved. 5 mm incisions were made on the backs of the mice. The bacteria-infected catheters were inserted subcutaneously and the incisions were then sealed with 3M Tegaderm. After 24 hrs, the mice were euthanized with CO₂. The implanted catheters were removed and the condition of the lumens assessed. The samples were then sonicated to remove the adhered bacteria, which were serially diluted and plated on LB agar plates. CFU were counted after 24 hrs of incubation at 37° C. The tests were performed one time with 5 independent biological replicates.

Results

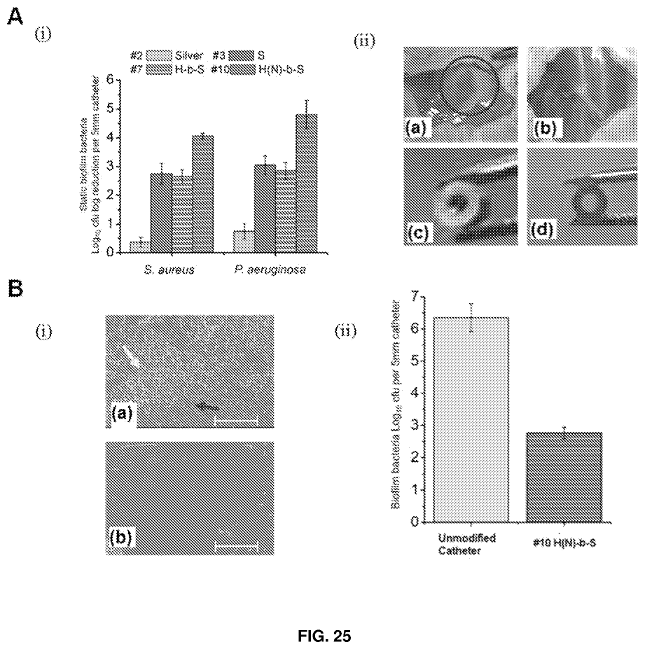

In the initial study, unmodified catheter (#1) and modified catheters (#10, #2, #3 and #7) were infected with high concentrations of MRSA-BAA38 and PAO1 (10⁷ cfu/mL) and implanted into a subcutaneous pocket created on mice. After 24 hours of implantation, the catheters were removed. The #10 H(N)-b-S catheter surface achieved an excellent (>4 log₁₀) bacteria adhesion reduction compared with the unmodified PU control catheter (FIG. 25Ai). Less than 1 log₁₀, 3 log₁₀ and 3 log₁₀ orders reduction was obtained with silver, #3, and #7 controls, respectively, against both S. aureus and P. aeruginosa.

The pocket implanted with unmodified PU-control catheter showed a yellowish mass which is indicative of infection or inflammation (FIG. 25A iia) and the intraluminal space of the catheter was filled with clotted subcutaneous blood (FIG. 25A iic). The pocket implanted with H(N)-b-S catheter (#10) was almost clean and showed no inflammation (FIG. 25A iib) and no clotted blood was observed in the intraluminal space of the catheter (FIG. 25A iid).

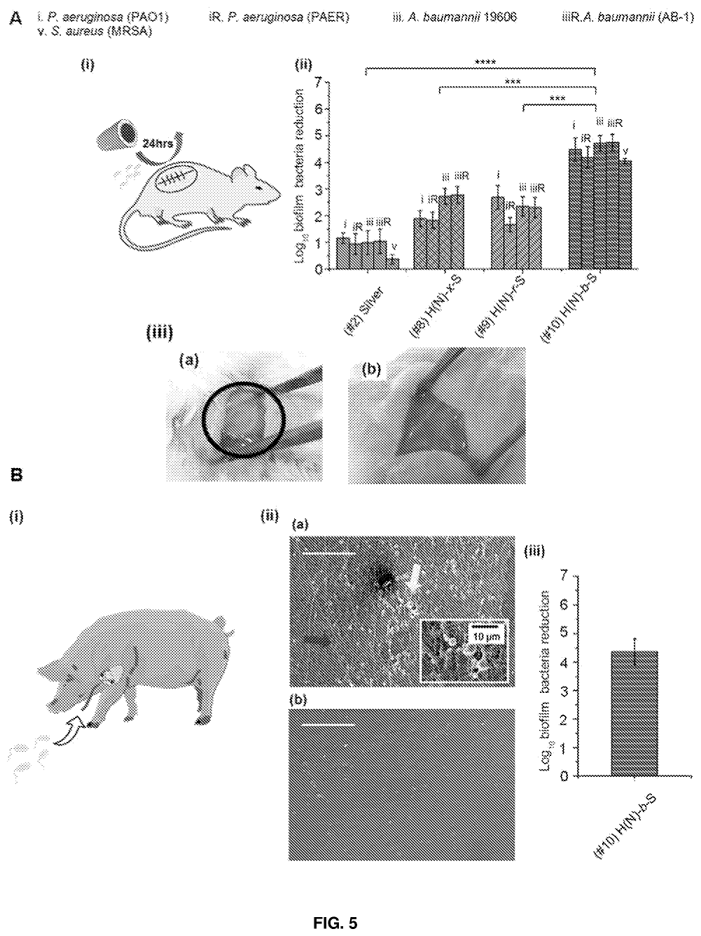

In the final study, the NO-releasing catheters (#8) H(N)-x-S, (#9) H(N)-r-S, (#10) H(N)-b-S and the (#1) unmodified catheter control were infected with high concentrations of P. aeruginosa or A. baumannii (10⁶ CFU/mL) and then implanted into subcutaneous pockets created on the backs of mice (FIG. 5A(i)). Both antibiotic-sensitive and antibiotic-resistant P. aeruginosa (PAO1 and PAER) or A. baumannii (ATCC 19606 and AB-1) were investigated. 24 hrs after implantation, the catheters were removed. The (#10) H(N)-b-S coated catheter achieved excellent (>4.0 log₁₀) antibiofilm effect against both P. aeruginosa and A. baumannii compared with less than 1.0 log₁₀ inhibition with silver (#2 (FIG. 5A(ii)). The (#10) H(N)-b-S also shows 1.8-2.6 log₁₀ better potency than the crosslinked hydrogel and random copolymer NO-releasing coatings ((#8) and (#9)) against P. aeruginosa and A. baumannii, including multi-drug resistant clinical isolates (FIG. 5A(ii)). The (#10) H(N)-b-S coated catheter also achieved excellent (4.1 log₁₀) antibiofilm effect against Gram-positive MRSA compared with less than 1.0 log₁₀ inhibition with silver (#2) (FIG. 5A(ii)).

The pocket implanted with (#1) unmodified PU control catheter showed a yellowish mass of pus which is indicative of infection and inflammation (FIG. 5A(iii)a, circle) while the pocket implanted with (#10) H(N)-b-S catheter was almost clean and without any visible signs of inflammation (FIG. 5A(iii)b).

### Example 11: In Vivo Porcine Haemodialysis Catheterization Model

(#10) H(N)-b-S coated catheters were tested in a porcine central venous catheterization (CVC) model (Journal of Controlled Release 2016, 241, 125) (FIG. 5B).

Method

The experiment was done in Beijing Maidisiwei Biotechnique service centre following ethical protocol no. 202001094 approved by Northwestern Polytechnical University, China. Bama miniature pigs were utilized for porcine model studies. The recommended normal mean artery pressure (MAP) range is 84-107 mmHg and the heart rate is 60-90 times per minute for a Bama miniature pig.

(a) Biocompatibility Test

The jugular vein of Pig (II) was implanted with the modified catheter (with (#10) H(N)-b-S coating), while the jugular vein of Pig (I) was implanted with unmodified PU catheter to provide a control. The jugular vein of only one side of Pig (I) or Pig (II) was implanted with one of the catheters. The Bama miniature pig (50 kg) was anesthetized with an intramuscular injection of 4 mL of zoletil-50 (25 mg/mL of zolazepam and 25 mg/mL of tiletamine) and 100 mg of xylazine. Inhalation of isoflurane (0.5-2% in pure oxygen) was maintained throughout the surgery. The pig was placed in a supine position. The neck was shaved and the skin of the surgical area was prepared with alcohol-based chlorhexidine and sterile dressings. The left jugular vein of the pig was exposed through standard open surgical techniques using a 5 cm skin incision. Before implantation, the catheters were flushed and washed in sterile isotonic saline. Each catheter was inserted by the Seldinger technique. After insertion of the 10 cm-long catheter, the catheter was plugged. The wound and catheter were covered with sterile dressing. Butorphanol was provided as a painkiller at 3 doses per day. Biomarkers such as blood pressure, heart rate, breath rate, blood oxygen level were measured by a tail cuff and the skin temperature was monitored by infra-red thermometer throughout 7 days of implantation. The test was performed one time with 1 independent biological replicate.

(b) Antibiofilm Test with Introduction of Bacterial Infection

Bama miniature Pig III (50 kg) was anesthetized with an intramuscular injection of 4 mL of zoletil-50 (25 mg/mL of zolazepam and 25 mg/mL of tiletamine) and 100 mg of xylazine. Inhalation of isoflurane (0.5-2% in pure oxygen) was maintained throughout the surgery. The pig was placed in a supine position. The neck was shaved, and the skin of the surgical area was prepared with alcohol-based chlorhexidine and sterile dressings. The bilateral jugular veins were exposed through standard open surgical techniques using a 5 cm skin incision. The unmodified catheter control (#1) and modified catheter (#10) were implanted in the left and right jugular veins respectively of the same pig for unbiased comparison. Before implantation, the catheters were flushed and washed in sterile isotonic saline. The catheters were implanted in the jugular vein by Seldinger technique (unmodified catheter on left side and modified catheter on right side), the distal end of each 10 cm-catheter was immersed in a MRSA (BAA-40) or (BAA-38) suspension of 2×10⁷ CFU/mL for 1 min and the same distal end was then pushed completely into the jugular vein. The catheters were then plugged. The wounds and catheters were covered with sterile dressings. Biomarkers such as blood pressure, heart rate, breath rate, blood oxygen level were measured by tail cuff and skin temperature was monitored by infra-red thermometer throughout implantation. No antibiotic was given before the surgery and during the experiment period. Butorphanol was used as a pain killer at 3 doses per day. After 5 or 7 days, the pigs were euthanized, and the implanted catheters were removed and cut into pieces. Bacteria adhesion and thrombus formation on implanted catheters were observed by Scanning Electron Microscopy, and bacteria adhesion on each piece of catheter was quantified by CFU counting. The test was performed one time with one independent biological replicate.

Results

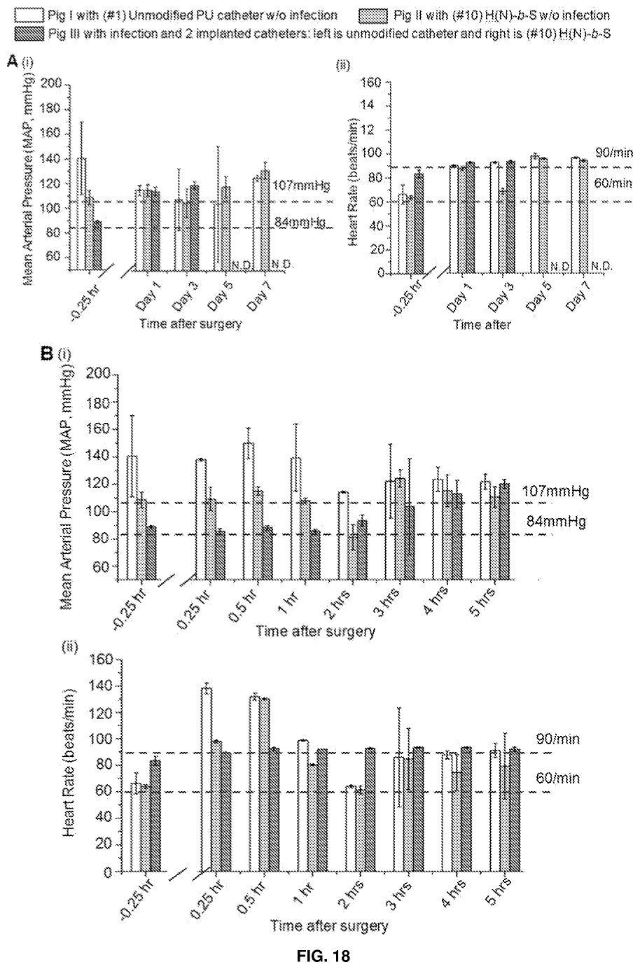

In respect of the in vivo biocompatibility of the modified catheter (#10) compared to uncoated catheter (#1), there was no significant difference in the mean arterial blood pressure (MAP) of Pig I versus Pig II on the 1^{st} day, 3^{rd} day, 5^{th} day and 7^{th} day after catheter implantation (FIG. 18Ai) (After surgery, the pigs were no longer under anaesthesia. The heart rates (HR) of both (uninfected) pigs were also in the normal range. These results indicate that the NO-releasing catheter (#10 H(N)-b-S) posed no long-term systemic toxicity to the pigs (FIG. 18Aii).

Pig III was implanted with 2 catheters that were both dosed with MRSA bacteria (the left jugular vein with (#1) unmodified catheter and the right jugular vein with #10 coated catheter). The long-term blood pressure (MAP) and heart rate (HR) of the infected Pig III were also normal (FIG. 18Ai and ii). This pig was euthanized on day 5 to obtain the antibacterial efficacy results (FIG. 5B). Based on the results from the three pigs, the #10 coated catheters showed no long-term systemic toxicity (for Day 1 to Day 7).

However, when the pigs were recovering from anaesthesia (i.e. during the first 5 hours after implantation, FIG. 18B), a transient drop in MAP was observed. The MAPs of both uninfected Pig I and Pig II dropped immediately after surgery until the 2^{nd} hour. Although the measured MAP of (uninfected) Pig II implanted with (#10) H(N)-b-S catheter at the 2^{nd} hour time point (81.3±9.2 mmHg) was slightly below the normal lower limit of MAP (84 mmHg), the blood pressure recovered after that. The MAP of Pig III which was implanted with (infected) (#10) coated catheter during the anaesthesia period (FIG. 18B) was quite stable and above the lower normal limit. Although it is not possible to rule out the possibility of transient systemic toxicity of NO combined with anaesthesia, proper dosing of NO and anaesthesia or delayed NO release should ensure the transient safety of NO-donor coated catheters during surgery.

To further evaluate the in vivo antibiofilm efficacy of catheters, a 10 cm length of (#1) unmodified PU catheter and (#10) H(N)-b-S coated catheter were prepared and implanted in the left and right jugular veins, respectively, of the same pig (Pig III) for unbiased comparison.

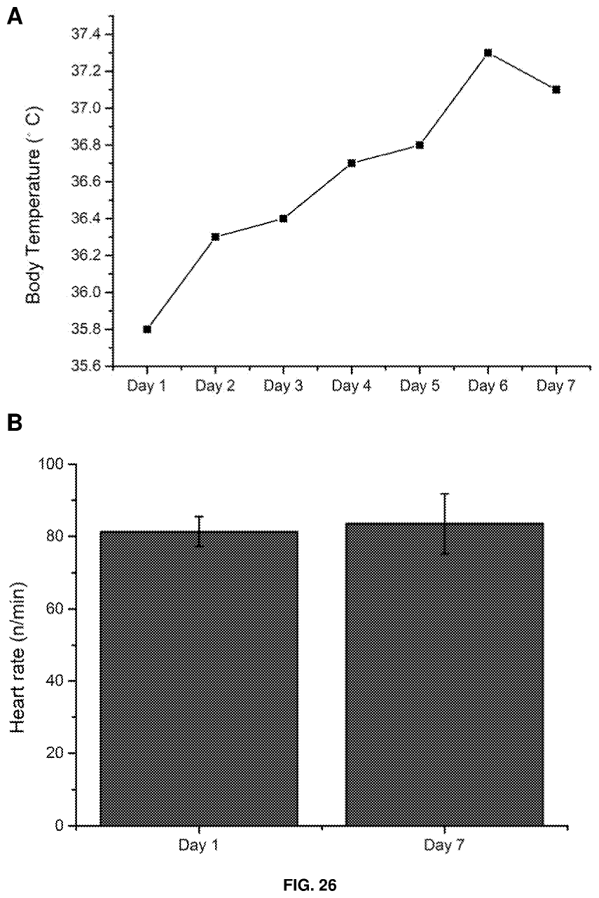

In the initial studies, the pig was infected with MRSA BAA-38 (10⁷ cfu/mL) after implantation and the body temperature was monitored. After 7 days of implantation, the implanted catheters were taken out and evaluated under scanning electron microscopy (SEM) (FIG. 25B(i)). The inner surface of the unmodified control catheter (FIG. 25B(i)a) was covered by MRSA biofilm (left arrow) and thrombus (right arrow). The #10 H(N)-b-S (FIG. 25B(i)b) catheter showed a clean surface without any trace of biofilm or development of thrombus. The amount of bacteria adhering on the surface of the catheters was quantified (FIG. 25B(ii)). The #10 H(N)-b-S catheter showed >3 log₁₀ reduction of bacteria adhesion compared with the unmodified catheter. The heart rate of the pig before and after implantation was not changed suggesting biocompatibility of #10 H(N)-b-S catheter (FIG. 26).

In the final studies, MRSA (BAA-40) was introduced to each catheter by dipping the external catheter terminal into bacteria inoculum in PBS (2×10⁷ CFU/mL) (FIG. 5B(i)). The catheter was then plugged, and the wound and catheter were covered with sterile dressings. After 5 days of implantation, the pig was sacrificed, and the implanted catheters were removed and evaluated with SEM (FIG. 5B(ii)). The inner surface of (#1) unmodified catheter (FIG. 5B(ii)a) showed significant adhesion of MRSA (sub-figure) and thrombi formation with activation of fibrinogen (arrow on left side) while (#10) H(N)-b-S coated catheter (FIG. 5B(ii)b) showed clean surface without any trace of biofilm or thrombus development and fibrinogen adhesion. The number of bacteria colonizing the surfaces of the catheters were quantified (FIG. 5B(iii)). The (#10) H(N)-b-S coated catheter showed 4.4 log₁₀ reduction of biofilm bacteria compared with (#1) unmodified PU catheter. The (#10) H(N)-b-S coated catheter showed excellent in vivo antibiofilm, anti-thrombus formation efficacy and biocompatibility.

Discussion of Results from Examples 1 to 11

The bacteria that infect medical devices in clinical settings are broad spectrum and it is important that the coating exhibits antimicrobial effect against this group of bacteria. It was shown that (#10) H(N)-b-S diblock polymer brush covalently grafted from PU catheter surface provided excellent short-term and long-term in vitro broad-spectrum antibiofilm activity against Gram-positive and Gram-negative bacteria. For the 24 hrs in vitro test, it achieved 99.99% to 99.999% (4.0-5.0 log₁₀) reduction of all seven Gram-positive and Gram-negative bacteria tested. In contrast, all other coatings, including (#2) silver and (#3) zwitterionic polymer brushes provided less than 99.9% (3.0 log₁₀) reduction against the same bacteria. In a murine subcutaneous infection model, (#10) coating achieved 99.9955% (4.1 log₁₀) and 99.9973% (4.8 log₁₀) reduction of MRSA and P. aeruginosa biofilms, respectively, while (#2) silver catheter provided 68% and 84% (0.5 and 0.8 log₁₀) reduction of these two bacteria. Further, in a porcine CVC infection model, (#10) coating achieved excellent in vivo medium-term antibiofilm effect of greater than 99.99% (4.4 log₁₀) inhibition of MRSA after 5-day implantation with good biocompatibility (FIG. 3, FIG. 4 and FIG. 5).

Against the Gram-positive bacteria, the NO-emitting coatings (#4, #8, #9 and #10) showed >3.0 log₁₀ inhibition. However, against the four Gram-negative bacteria, coating (#10) stood out as distinctly superior to the other coatings, with >4.7 log₁₀ inhibition against all tested pathogens. Some of the other coatings approach this performance against individual pathogens, but against the group none is nearly as effective as (#10). The non-NO-emitting coatings ((#3) S, (#5) H-x-S, (#6) H-r-S and (#7) H-b-S) showed inferior antibiofilm activity (1.8-2.9 log₁₀ inhibition) against all bacteria tested compared with (#10) (4.4-5.3 log₁₀) (FIG. 15A). Of the 3 differently structured NO-releasing coatings (#8, #9, #10), only the precision diblock copolymer (#10) has good broad spectrum antibiofilm effect with excellent blood compatibility (FIGS. 3 and 4). The crosslinked and random copolymer brush coatings ((#8) and (#9), FIGS. 12 and 13), which do not sequester the NO-release agent below the surface, show poor antibiofilm effect against some Gram-negative bacteria, higher activation of immune cells and more fouling with blood protein.

The covalent attachment of RSNO to the grafted brush coating prevented leaching of RSNO (FIG. 2E) and results in sustained NO flux over 15 days (FIG. 2F), which is about one order of magnitude higher than reported values with blending (ACS Biomaterials Science & Engineering 2015, 1, 416). Blending of RSNO with a polymer coating without covalent bonding would result in leaching with ensuing toxicity and a shorter period of sustained release. Both crosslinked and random copolymer brush coatings ((#8) and (#9)), which do not sequester the NO-release agent below the outer surface, are more hydrophobic than their non-RSNO-functionalized counterparts ((#5) and (#6), as shown by their water contact angle measurements. The sequestration of hydrophobic RSNO in the subsurface block of (#10) H(N)-b-S did not reduce the hydrophilicity of the outer hydrophilic poly(SBMA) block as shown by its still small contact angle (8.0±0.4°, FIG. 2D). The combination of surface hydrophilicity and NO emission is necessary to effectively inhibit biofilm by a broad spectrum of pathogens.

Further, the (#8) crosslinked and (#9) random copolymer coatings made by free-radical copolymerization have much higher roughness than (#10) block copolymer brush coatings. The RSNO decomposes to thiyl radicals (RS.) after the release of the NO molecule which may reduce the biocompatibility of the coating (Free Radical Biology and Medicine 1989, 7(6), 659; Methods in Enzymology 1995, 251, 117). The disulphide bonds formed from two adjacent RSNO molecules which released their NO molecules may remain bio-active toward thiol-containing proteins (Acta Biomaterialia 2016, 29, 446). The coatings (#8) and (#9) may expose these radicals or disulphide bonds which would activate immune cells (FIG. 4C) or cause protein fouling (FIG. 4D). In contrast, the block copolymer coating with a hydrophilic layer (#10) sequesters the thiyl radicals (RS.) and disulphide bond to prevent activation of immune cells or protein fouling (FIGS. 4C and D). The higher hydrophilicity, lower surface roughness and reduced surface bioactivity of (#10) prevent blood protein fouling and thrombus formation, reduces immune activation and attachment of bacteria and proteins. The sequestration of RSNO in the subsurface block in (#10) is important to the multifunctionality of this coating.

The (#10) diblock copolymer was made by an ozone-initiated surface RAFT technique. The ozone treatment provides a high density of peroxide initiator groups (169 peroxide initiators/nm²) on both the inner and outer surfaces of catheters in spite of their narrow and long bores. The O₃ treatment results in significantly denser initiating points compared with other chemistries such as thiol-Au linkage, silane chemistry and plasma activation. For example, plasma activation (Polymer Chemistry 31, 1035-1043 (1993)) was reported to introduce 10-15 surface peroxide groups/nm². Silane chemistry typically achieves 0.04-4 groups/nm² (Macromolecules 33, 5608-5612 (2000)). The high-density peroxide/hydroperoxide is probably not formed as a single layer but as a multi-layer structure as O₃ gas diffuses through. The O₃ treatment can also be applied on different kinds of substrates, including different plastics such as silicone, polyurethane (PU), poly(methyl methacrylate (PMMA) and polyethylene (PE).

In addition, the surface-initiated polymer brush is chemically bonded to the catheter material which is a more robust attachment than previously reported initiator impregnation methods which result in unstable brush coating (as evidenced by reversion of contact angles after 14 days; Chemistry Communications 2016, 52). The ozone-surface-RAFT technique provides a uniform, dense and precisely structured block copolymer brush which achieves outstanding functionality. It is readily applicable to the surface modification of catheters at scale and, more generally, is applicable to the formation of controlled multifunctional coatings on complex and relatively inaccessible surfaces of a wide range of medical devices.

Conclusion of Examples 1 to 11

A diblock brush coating (#10) H(N)-b-S was shown to achieve excellent short-term and long-term antibiofilm effect against Gram-negative and Gram-positive bacteria with excellent blood compatibility. It outperforms previous coatings which are purely hydrophilic or contain an exposed NO-donor. The precise placement of the surface antifouling block and the subsurface NO-emitting block is critical for achieving excellent antibiofilm effect with excellent thrombus formation resistance and biocompatibility. The (#10) H(N)-b-S coating achieved excellent 4.4-5.3 log₁₀ short term (24 hrs) inhibition of biofilm formation in 7 clinically relevant Gram-positive and Gram-negative bacteria and outperformed (#2) silver and a hydrophilic coating (#7) by around 4.0 log₁₀ and 2.0 log₁₀, respectively. The (#10) H(N)-b-S is the first report of a catheter coating that is shown to impart long-term antibiofilm activity to catheters of clinically useful lengths (30 cm), achieving 3.0 log₁₀ reduction of MRSA and P. aeruginosa biofilms at the 30th day in an intraluminal in vitro model. In a murine subcutaneous catheter infection model, this coating achieves greater than 4.0 log₁₀ inhibition of Gram-positive MRSA and Gram-negative P. aeruginosa, and A. baumannii respectively. Further, the catheter coating is shown to provide excellent antibiofilm efficacy in an in vivo central venous infection model.

The ozone-surface-RAFT coating method developed in this work shows great potential for the achievement of high-performance anti-infective coatings on other medical devices besides catheters, which require coatings which are multifunctional, non-leaching and long-term biofilm-resistant. Similar to catheters, it is difficult to provide a uniform coating on such devices. The H(N)-b-S coating may have a transformative impact in reducing infections associated with catheter use in healthcare settings and in a wide range of biomedical devices, including those of structures which are difficult to be modified using conventional techniques.

### Reference Example 12: Model System for Fabricating a Stable and Long-Term NO Releasing Catheter

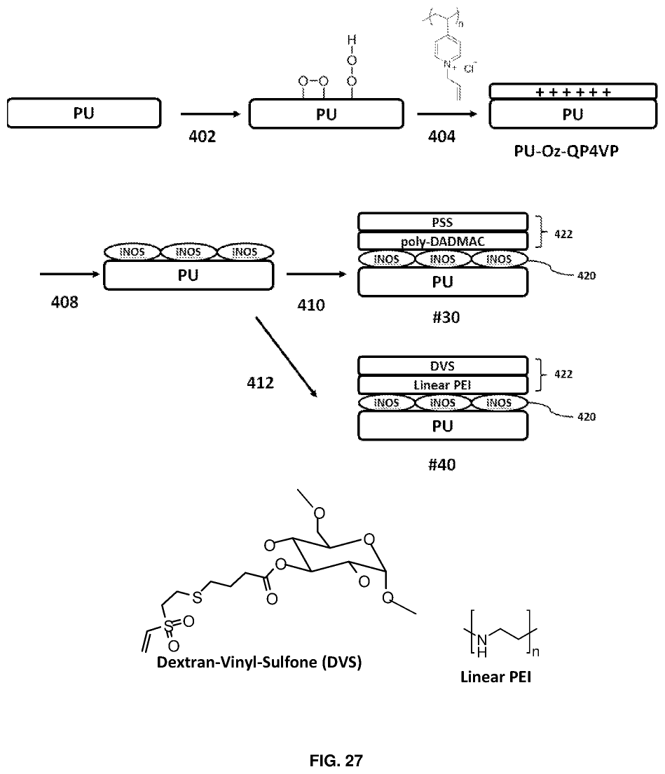

In this example, two NO releasing catheters (#30 and #40) were fabricated via a layer by layer (LBL) coating technique (FIG. 27). This example shows that an enzyme may be conjugated to a polymeric material and then used as a nitric oxide source.

Fabricating an Electrostatic LBL Coating #30

Peroxide groups were first introduced on a polyurethane catheter surface by ozone treatment following General Procedure 1 (step 402). The ozone-treated catheter was then dipped into a solution comprising of 5% quaternized poly(vinyl)pyridinium chloride (QP4VP) in methanol, and taken out. The dip-coated catheter was heated to 100° C. to provide a coating of solidified quaternized poly(vinyl)pyridinium chloride (QP4VP) on the catheter surface (step 404). This coating has a positive charge. An inducible isoform of the enzyme nitric oxide synthase (iNOS) was then introduced on the catheter by LBL technique (step 408). The nitric oxide synthase is anionically charged. The catheter coated with cationic charged QP4VP was immersed in 10% iNOS solution for 10 mins for attachment of enzyme by electrostatic interaction. Then, the catheter was washed by DI water. The surface of the catheter at this step is anionically charged.

Subsequently, polydiallyldimethylammonium chloride (poly-DADMAC), followed by poly(sodium 4-styrenesulfonate) (PSS) were introduced to the product of step 408 by LBL technique (step 410). Specifically, the anionically charged catheter was firstly immersed in poly-DADMAC (10 w/w % in DI water) for 10 mins for electrostatic attachment of the cationic polymer. After the attachment, the catheter was cationically charged and transferred to anionic PSS solution. The cycle was repeated a few times to achieve a robust coating.

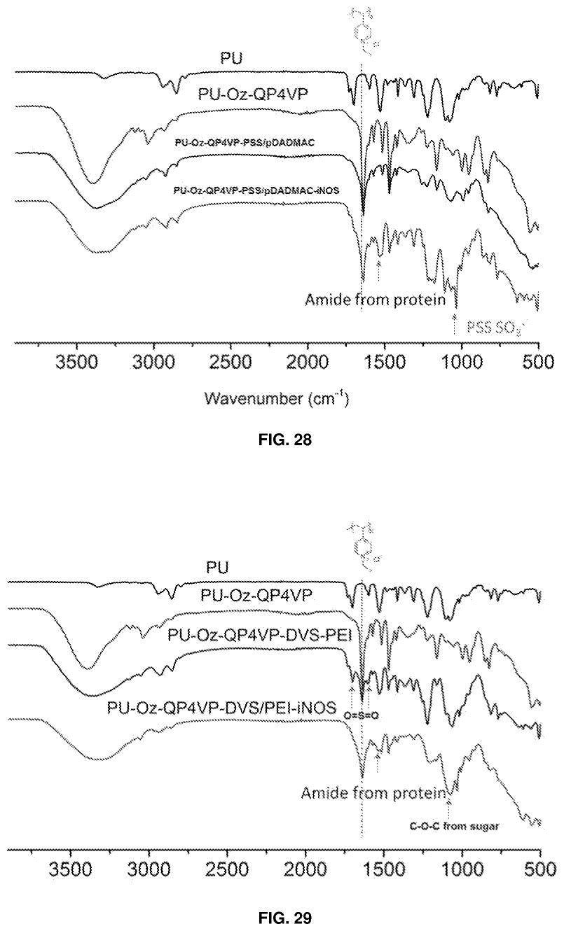

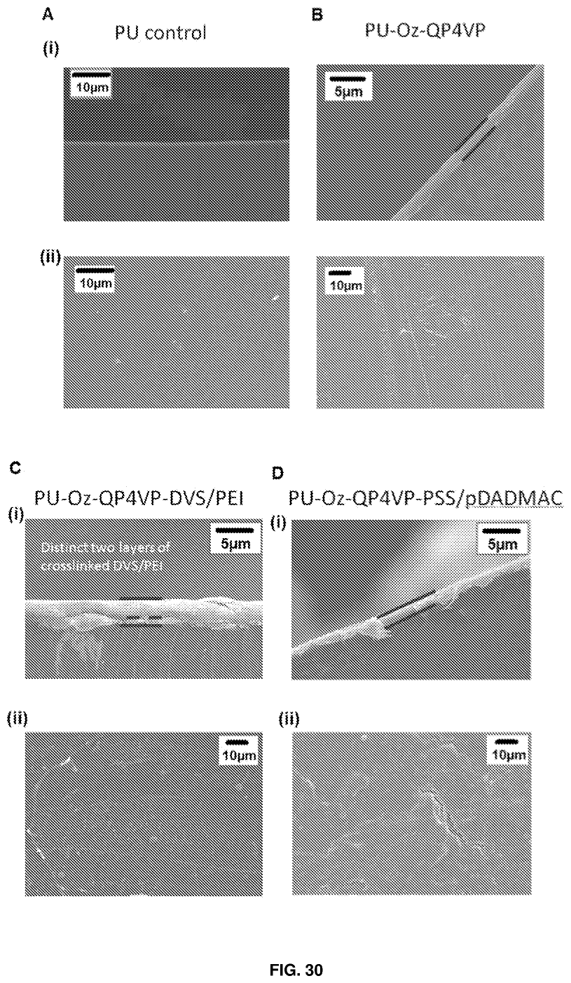

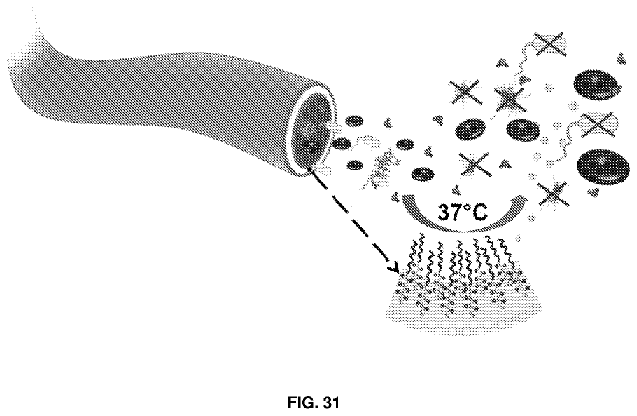

This provides a final coating #30 (PU-Oz-QP4VP-PSS/pDADMAC-iNOS), comprising of a NO releasing layer (420) formed from iNOS attached as a bottom layer closest to the PU substrate and an antifouling hydrophilic layer (422) attached as a top layer above the NO-releasing layer. The surface morphology was tracked by FTIR (FIG. 28) and was observed using SEM (FIG. 30D). The water contact angle was measured to be 10.4°.

Fabricating a Covalent LBL Coating #40

Linear polyethylenimine (PEI), followed by a dextran-vinyl-sulfone (DVS) was introduced to the product of step 408 by LBL technique in accordance with the procedure for forming coating #30 with changes to the reactants (step 412). This provides a final coating #40 (PU-Oz-QP4VP-DVS/PEI-iNOS). It comprises of a NO releasing layer (420) formed from iNOS attached as a bottom layer closest to the substrate and an antifouling hydrophilic layer (422) attached as a top layer above the NO-releasing layer. The surface morphology was tracked by FTIR (FIG. 29) and was observed using SEM (FIG. 30C). The water contact angle was measured to be 21.6°.

Antibiofilm Efficacy Against MRSA

The antibiofilm efficacy of the catheters was measured following the procedure set out in Example 5. #30 (PU-Oz-QP4VP-PSS/pDADMAC-iNOS) achieved a log reduction of 3.57. #40 (PU-Oz-QP4VP-DVS/PEI-iNOS) achieved a log reduction of 4.2 after incubation at 55° C. to simulate ethylene oxide sterilization.

1-24. (canceled)

25. A composite material comprising a substrate coated with a block copolymer brush, where the block copolymer brush comprises:
a first block of a hydrophobic polymer conjugated to a nitric oxide source, where the first block of the hydrophobic polymer is covalently bonded to a surface of the substrate or a first block of a cationic polymer covalently bonded to a surface of the substrate; and
a second block of a hydrophilic polymer, extending from the first block to form an outer surface of the block copolymer brush.

26. The composite material according to claim 25, wherein the composite material further comprises a nitric oxide source conjugated to the cationic polymer of the first block.

27. The composite material according to claim 25, wherein the nitric oxide source is selected from one or more of a S-nitrosothiol, a NONOate, and a nitric oxide synthase.

28. The composite material according to claim 27, wherein the nitric oxide source is one or both of a S-nitrosothiol and a nitric oxide synthase.

29. The composite material according to claim 28, wherein the nitric oxide source is a tertiary S-nitrosothiol.

30. The composite material according to claim 25, wherein the block copolymer brush coating is from 0.1 to 20 μm thick.

31. The composite material according to claim 25, wherein the substrate is formed from a polymeric material selected from one or more of the group consisting of polyurethane, silicone, latex, and polyester polyurethane.

32. The composite material according to claim 31, wherein the substrate is formed from polyurethane.

33. The composite material according to claim 25 wherein the first block is formed from a polymer comprising one or more vinylogous monomers, where at least one of the one or more vinylogous monomers contains a functional group suitable to conjugate to a nitric oxide source.

34. The composite material according to claim 33, wherein the one or more vinylogous monomers are selected from the group consisting of an acrylate, an alkylacrylate, an acrylamide, an alkylacrylamide, an acrylic acid, an alkyl acrylic acid, and an allylic monomer compound.

35. The composite material according to claim 34, wherein the one or more vinylogous monomers are selected from the group consisting of allyl amine, amino ethyl methacrylate (AEMA), hydroxyl ethyl methacrylate (HEMA), hydroxylethyl acrylate (HEA), hydroxyl ethyl acrylamide (HEAA), hydroxypropyl methacrylate, hydroxybutyl acrylate, N-[Tris(hydroxymethyl)methyl] acrylamide, allyl alcohol, acrylic acid, methacrylic acid, glycidyl methacrylate (GMA), and ally glycidyl ether (AGE).

36. The composite material according to claim 33, wherein the first block is formed from a polyacrylate.

37. The composite material according to claim 25, wherein the second block is formed from a polymer comprising one or more vinylogous hydrophilic monomers.

38. The composite material according to claim 37, wherein the one or more vinylogous hydrophilic monomers are selected from the group consisting of a hydrophilic acrylate, a hydrophilic alkylacrylate, a hydrophilic acrylamide, a hydrophilic alkylacrylamide, a hydrophilic acrylic acid, a hydrophilic alkyl acrylic acid, and a hydrophilic allylic monomer compound.

39. The composite material according to claim 25, wherein:
the first block is formed from poly(hydroxyethyl methacrylate);
the second block is formed from poly(sulfobetaine methacrylate); and
the nitric oxide source is a tertiary S-nitrosothiol.

40. A method of forming a composite material as described in claim 25, the method comprising the steps of:
(a) providing a substrate coated with a polymer brush, where the polymer brush is formed from a hydrophobic or cationic polymer covalently bonded to a surface of the substrate;
(b) reacting the hydrophobic or cationic polymer in the polymer brush with a hydrophilic monomer in a polymerisation reaction to form a block copolymer brush; and
(c) conjugating a nitric oxide source to the hydrophobic or cationic polymer of the block copolymer brush.

41. A method of forming a composite material as described in claim 25, said method comprising the steps of:
(ai) providing a substrate coated with a block copolymer brush, where the block copolymer brush is formed from:
a first block of a hydrophobic or cationic polymer covalently bonded to a surface of the substrate; and
a second block of a hydrophilic polymer, extending from the first block to form an outer surface of the block copolymer brush; and
(bi) conjugating a nitric oxide source to the hydrophobic or cationic polymer of the first block.

42. An article formed from a composite material as described in claim 25, wherein a substrate of the composite material has at least one surface that is coated with a block copolymer brush as described in claim 25.

43. The article according to claim 42, wherein the article is a catheter formed from the composite material, where the catheter has an outer surface and an inner surface, and one or both of the outer surface and inner surface have been coated with the block copolymer brush.

44. The article according to claim 42, wherein the article is a wound dressing formed from the composite material, where the wound dressing has an outer surface and an inner surface, and the inner surface is coated with the block copolymer brush.
